(19) United States
(12) Patent Application Publication
(10) Pub. No.: US 2002/0182626 A1
(43) Pub. Date: Dec. 5, 2002

(54) Episomal non-transforming nucleic acid elements in functional genomic and antigenic applications

(76) Inventor: Daniel Tuse, Vacaville, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/098,606

(22) Filed: Mar. 15, 2002

Related U.S. Application Data
(60) Provisional application No. 60/276,886, filed on Mar. 16, 2001.

(51) Int. Cl. C12Q 1/68; G01N 33/53; C12N 15/87

(52) U.S. Cl. 435/6; 435/7.1; 435/455

(57) ABSTRACT

The present invention provides a method for functional genomic analysis and antigen prototyping by introducing episomal non-transforming non-viral vector-based cDNA or genomic libraries of donor organisms, pathogens, or cancer cells into host cells. The DNA libraries can be compacted without substantial aggregation to facilitate uptake by host cells. Antigen prototyping can be to identify antigens of pathogens that confer immuno-protection against the pathogens and antigens of cancer cells that confer immuno-protection against the cancer cells..

### CROSS-REFERENCE TO RELATED APPLICATION

The present invention is a continuation of U.S. Provisional Application Serial No. 60/276,886, filed Mar. 16, 2001.

### FIELD OF THE INVENTION

The present invention relates generally to the field of molecular biology and genetics. Specifically, the present invention relates to a method using episomal non-transforming nucleic acid replicating elements for determining the presence of a trait in a host organism, for antigen prototyping, and cancer antigen prototyping.

### BACKGROUND OF THE INVENTION

The expression of a foreign gene in a host cell is generally achieved by transferring the gene into the host cell using a gene transfer vector. Gene transfer vectors are available in the art and include for example retrovirus vectors, adenovirus vectors, and adeno-associated viral vectors. Many transfer vectors operate by integrating at least the transferred gene, if not the complete gene transfer vectors, into the host cell chromosome, although non-integrating transfer vectors are known in the art. The efficiency of stable integration of transfected gene constructs is generally very inefficient. The disadvantage of using viral vector-based gene transfer is that the amount of genetic material that the vector is able to accommodate is limited by the genetic material packaging limitations of the virus. Gene transfer vectors that include large fragments of inserted genetic material are difficult to produce at a sufficiently high titre to be of practical value. Therefore, it is difficult to include additional genetic material in a virus-based vector, for example, gene regulatory elements, without deleteriously affecting stable gene transfer.

Work has been conducted in the area of developing suitable vectors for expressing foreign DNA and RNA in animal hosts. Ahlquist, U.S. Pat. Nos. 4,885,248 and 5,173,410 describes preliminary work done in devising transfer vectors which might be useful in transferring foreign genetic material into a host organism for the purpose of expression therein. All patent references cited herein are hereby incorporated by reference. Additional aspects of hybrid RNA viruses and RNA transformation vectors are described by Ahlquist et al. in U.S. Pat. Nos. 5,466,788, 5,602,242, 5,627,060 and 5,500,360, all of which are incorporated herein by reference. Condreay et al. (Proc. Natl. Acad. Sci. USA, 96:127-132) (1999) disclose using baculoviruses to deliver and express gene efficiently in cells types of human, primate and rodent origin. Price et al., Proc. Natl. Acad. Sci. USA, 93:9465-9570 (1996)) disclose infecting insect, plant and mammalian cells with Nodaviruses.

Methods and compositions have been described consisting of viral vectors that replicate cytoplasmically and that contain plus-sense inserts to yield polypeptide gene products (biomanufacturing), as well as same or similar viral vectors that replicate cytoplasmically and that expresses libraries of genes inserted in plus or minus sense orientation, to yield gene functional information (functional genomics). This art is described in issued and pending patent applications generally referred to as “GENEWARE” technology of the Large Scale Biology Corp. (Vacaville, Calif.); generally consists of RNA viral vectors replicating in plant hosts (see U.S. Pat. No. 5,922,602). Non-transforming DNA viral vectors (e.g., circavirus replicons), with the ability to replicate in animal, including human cells lines, have also been described. Library expression of genes in such viral vectors, in plus sense or antisense orientation, could yield functional genomic information in animal, including human, host cells generally referred to as “HUMAN GENEWARE” technology (see U.S. Pat. No. 6,054,566).

Notwithstanding the advantages of using viral replicons to achieve the above mentioned utilities, including potentially high copy number as well as systemic translocation in vivo, disadvantages also exist, especially in functional genomic and antigenic applications. Such disadvantages may include (1) perturbation of host proteins, especially at high copy number, which may significantly alter the normal phenotype of the host and complicate interpretation of gene function results, especially when several hosts are used. Also, (2) host specificity may narrow the utility of viral replicons.

To overcome such limitations, “naked” non-viral DNA replicons, have been described, which, when introduced into competent host tissue, can enter the nucleus and replicate. By normal transcriptional and translational processes, these vectors can encode the synthesis of a wide variety of proteins. Some of the art describes the use of such naked DNA plasmids to produce proteins in vivos that are recognized as antigens by the host, thereby triggering an immune response (eg., genetic vaccination, or gene vaccines, etc.). Such technology forms the intellectual property portfolio of companies such as Vical, Inc. (San Diego, Calif.) (see, e.g. U.S. Pat. Nos. 5,910,488 and 6,147,055).

While naked plasmid DNA containing functional exogenous genes has been introduced into competent host cells in vitro via a variety of physical techniques, including transfection, direct microinjection, electroporation, and coprecipitation with calcium phosphate, most of these methods are impractical for delivering genes to cells within intact animals. Furthermore, even in highly controllable in vitro applications, naked plasmid DNA, by virtue of its (1) poor uptake, (2) susceptibility to degradation, or (3) low copy number, may not make an ideal vector for either gene therapy, functional genomic, or antigenic applications. Likewise, hybrid DNA viral replicons, by virtue of their narrow host range, toxicity/pathogenicity to the host, antigenicity of viral proteins, or low copy number within desired host, may also not be ideal vector for gene therapy, functional genomic or antigenic applications.

There are disclosures that describe the use of non-viral episomal DNA replicons as non-transforming gene therapy vectors. Vectors consisting of a papovavirus origin of replication and a mutant form of a papovavirus large T antigen are replication-competent and transformation-negative. Because such vectors can achieve high copy number, they can be used to effect gene therapy (eg., antisense) in competent malignant host tissue (U.S. Pat. No. 5,624,820). These vectors can be designed to operate upon entry into host nuclei, or regulated to become activated constitutively upon an environmental signal, such as a hormonal signal, thereby limiting or controlling the extent of vector activity.

An improved type of non-viral nucleic acid replicon, consisting of viral promoters and other replication control signals, and exogenous genes in either plus or minus sense orientation, and capable of (1) high copy number and (2) substantially longer half-lives in the host, have been described (see U.S. Pat. Nos. 5,624,820; 5,844,107; 5,877,302; 5,972,900; 5,972,901; and 6,008,336). Such vectors can be seen as embodying the best aspects of both viral and non-viral replicons, which may endow them with improved utility over prior art in gene expression, functional genomic and antigenic applications.

These vectors have only been disclosed or suggested to allow the body (instead of factories) to make therapeutic proteins and to allow the body to replace defective receptors in specific organs. However, none of these patents anticipate, teach claim or suggest the use of such non-viral vector compositions in quantitative gene expression or function (functional genomic) or in antigen discovery or prototyping (antigenic) applications. The present invention describes methods to determine the function of exogenous genes (functional genomic) as well as methods to discover and prototype antigens (antigenics), using these compositions.

### SUMMARY OF THE INVENTION

The present invention provides for a method of compiling a functional gene profile of a donor organism by introducing an episomal non-transforming non-viral vector-based library of nucleic acid inserts from the donor organism. The present invention also provides for a method of identifying the sequence of an antigen of a pathogen, which when expressed in a host confers immuno-protection on the host against the pathogen by introducing an episomal non-transforming non-viral vector-based library of nucleic acid inserts from the pathogen. The present invention also provides for a method of identifying the sequence of an antigen of a cancer cell, which when expressed in a host confers immuno-protection on the host against the cancer cell. The method comprises introducing an episomal non-transforming non-viral vector-based library of nucleic acid inserts from the cancer cell.

One embodiment of the invention is a method of compiling a functional gene profile of a donor organism, comprising: (a) introducing into an episomal non-transforming non-viral vector a mixture of a donor organism derived DNA or RNA sequences to construct an episomal non-transforming non-viral vector-based library, wherein the sequences are unidentified, wherein each member of the library comprises an insert from the mixture; (b) introducing into a host said one or more members of the library; (c) transiently expressing said unidentified nucleic acid in the host; (d) determining one or more phenotypic or biochemical changes in the host; (e) identifying an associated trait relating to said one or more phenotypic or biochemical changes; (f) identifying the member that results in said one or more changes in the host; and (g) repeating steps (b)-(f) until at least one nucleic acid sequence associated with said trait is identified, whereby a functional gene profile of the host or of the donor organism is compiled.

Another embodiment of the invention is a method of identifying the sequence of an antigen of a pathogen, which when expressed in a host confers immuno-protection on the host against the pathogen, comprising: (a) introducing into an episomal non-transforming non-viral vector a mixture of the pathogen derived DNA or RNA sequences to construct an episomal non-transforming non-viral vector-based library, wherein each member of the library comprises an insert from the mixture; (b) introducing the library into a group of hosts wherein each host contains one member; (c) expressing each insert, capable of expression in the host, in the host in which the member resides; (d) challenging each of the host with the pathogen; (e) determining which host has immuno-protection against the pathogen; and (f) determining the sequence of the insert in the host determined in step (e); whereby the sequence of the antigen of the pathogen is identified.

Another embodiment of the invention is a method of identifying the sequence of an antigen of a cancer cell, which when expressed in a host confers immuno-protection on the host against the cancer cell, comprising: (a) introducing into an episomal non-transforming non-viral vector a mixture of the cancer cell derived DNA sequences to construct an episomal non-transforming non-viral vector-based library, wherein each member of the library comprises an insert from the mixture; (b) introducing the library into a group of hosts wherein each host contains one member; (c) expressing each insert, capable of expression in the host, in the host in which the member resides; (d) challenging each of the host with the cancer cell; (e) determining which host has immuno-protection against the cancer cell; and (f) determining the sequence of the insert in the host determined in step (e); whereby the sequence of the antigen of the cancer cell is identified.

### DESCRIPTION OF THE SPECIFIC EMBODIMENTS

### The Invention

The present invention provides for a method of compiling a functional gene profile of a donor organism by introducing an episomal non-transforming non-viral vector-based library of nucleic acid inserts from the donor organism. The present invention also provides for a method of identifying the sequence of an antigen of a pathogen, which when expressed in a host confers immuno-protection on the host against the pathogen by introducing an episomal non-transforming non-viral vector-based library of nucleic acid inserts from the pathogen. The present invention also provides for a method of identifying the sequence of an antigen of a cancer cell, which when expressed in a host confers immuno-protection on the host against the cancer cell. The method comprises introducing an episomal non-transforming non-viral vector-based library of nucleic acid inserts from the cancer cell.

One embodiment of the invention is a method of compiling a functional gene profile of a donor organism, comprising: (a) introducing into an episomal non-transforming non-viral vector a mixture of a donor organism derived DNA or RNA sequences to construct an episomal non-transforming non-viral vector-based library, wherein the sequences are unidentified, wherein each member of the library comprises an insert from the mixture; (b) introducing into a host said one or more members of the library; (c) transiently expressing said unidentified nucleic acid in the host; (d) determining one or more phenotypic or biochemical changes in the host; (e) identifying an associated trait relating to said one or more phenotypic or biochemical changes; (f) identifying the member that results in said one or more changes in the host; and (g) repeating steps (b)-(f) until at least one nucleic acid sequence associated with said trait is identified, whereby a functional gene profile of the host or of the donor organism is compiled.

Another embodiment of the invention is a method of identifying the sequence of an antigen of a pathogen, which when expressed in a host confers immuno-protection on the host against the pathogen, comprising: (a) introducing into an episomal non-transforming non-viral vector a mixture of the pathogen derived DNA or RNA sequences to construct an episomal non-transforming non-viral vector-based library, wherein each member of the library comprises an insert from the mixture; (b) introducing the library into a group of hosts wherein each host contains one member; (c) expressing each insert, capable of expression in the host, in the host in which the member resides; (d) challenging each of the host with the pathogen; (e) determining which host has immuno-protection against the pathogen; and (f) determining the sequence of the insert in the host determined in step (e); whereby the sequence of the antigen of the pathogen is identified.

Another embodiment of the invention is a method of identifying the sequence of an antigen of a cancer cell, which when expressed in a host confers immuno-protection on the host against the cancer cell, comprising: (a) introducing into an episomal non-transforming non-viral vector a mixture of the cancer cell derived DNA sequences to construct an episomal non-transforming non-viral vector-based library, wherein each member of the library comprises an insert from the mixture; (b) introducing the library into a group of hosts wherein each host contains one member; (c) expressing each insert, capable of expression in the host, in the host in which the member resides; (d) challenging each of the host with the cancer cell; (e) determining which host has immuno-protection against the cancer cell; and (f) determining the sequence of the insert in the host determined in step (e); whereby the sequence of the antigen of the cancer cell is identified.

The host may be a cell or a whole organism. The cell may be part of a cell culture, a tissue, a tissue culture, or an organ. The host may be any mammalian species, such as: one important for research purposes, such as a mouse, rat, hamster, rabbit, pig, goat, primate, or the like; or, one important for commercial purposes, such as a goat, sheep, cow, cattle, horse, pig, dog, cat, or the like. The host may also be a human. It will be noted that these animals come from four orders of class: Mammalia: Primata, Rodenta, Carnivora, and Artiodatyla. The host may be an animal oocyte, egg, embryo, embryonic stem cell, or any other specific animal tissue. The host may be transgenic or non-transgenic.

The donor organism and pathogen may be any organism of any classification, which includes Kingdom Monera, Kingdom Protista, Kingdom Fungi, Kingdom Plantae and Kingdom Animalia. Kingdom Monera includes subkingdom Archaebacteriobionta (archaebacteria): division Archaebacteriophyta (methane, salt and sulfolobus bacteria); subkingdom Eubacteriobionta (true bacteria): division Eubacteriophyta; subkingdom Viroids; and subkingdom Viruses. Kingdom Protista includes subkingdom Phycobionta: division Xanthophyta 275 (yellow-green algae), division Chrysophyta 400 (golden-brown algae), division Dinophyta (Pyrrhophyta) 1,000 (dinoflagellates), division Bacillariophyta 5,500 (diatoms), division Cryptophyta 74 (cryptophytes), division Haptophyta 250 (haptonema organisms), division Euglenophyta 550 (euglenoids), division Chlorophyta, class Chlorophyceae 10,000 (green algae), class Charophyceae 200 (stoneworts), division Phaeophyta 900 (brown algae), and division Rhodophyta 2,500 (red algae); subkingdom Mastigobionta 960: division Chytridiomycota 750 (chytrids), and division Oomycota (water molds) 475; subkingdom Myxobionta 320: division Acrasiomycota (cellular slime molds) 21, and division Myxomycota 500 (true slime molds). Kingdom Fungi includes division Zygomycota 570 (coenocytic fungi): subdivision Zygomycotina; and division Eumycota 350 (septate fungi): subdivision Ascomycotina 56,000 (cup fungi), subdivision Basidiomycotina 25,000 (club fungi), subdivision Deuteromycotina 22,000 (imperfect fungi), and subdivision Lichenes 13,500. Kingdom Plantae includes division Bryophyta, Hepatophyta, Anthocerophyta, Psilophyta, Lycophyta, Sphenophyta, Pterophyta, Coniferophyta, Cycadeophyta, Ginkgophyta, Gnetophyta and Anthophyta. Kingdom Animalia includes: Porifera (Sponges), Cnidaria (Jellyfishes), Ctenophora (Comb Jellies), Platyhelminthes (Flatworms), Nemertea (Proboscis Worms), Rotifera (Rotifers), Nematoda (Roundworms), Mollusca (Snails, Clams, Squid & Octopus), Onychophora (Velvet Worms), Annelida (Segmented Worms), Arthropoda (Spiders & Insects), Phoronida, Bryozoa (Bryozoans), Brachiopoda (Lamp Shells), Echinodermata (Sea Urchins & starfish), and Chordata (Vertebrata-Fish, Birds, Reptiles, Mammals). A preferred donor organism is human. Donor organisms and pathogens include organisms from Monera, Protista, Fungi and Animalia. The donor organism and pathogen may also be any virus. Organisms that are pathogens are any organisms that cause disease in the specific host used. For example, pathogens for a human include bacteria, viruses, fungi, protozoan, roundworm, flatworm, louse, mite, sandflea, and the like.

To prepare a DNA insert comprising a nucleic acid sequence of a donor organism, the first step is to construct a cDNA library, a genomic DNA library, or a pool of mRNA of the donor organism. Full-length cDNAs or genomic DNA can be obtained from public or private repositories. For example, cDNA and genomic libraries from bovine, chicken, dog, drosophila, fish, frog, human, mouse, porcine, rabbit, rat, and yeast; and retroviral libraries can be obtained from Clontech (Palo Alto, Calif.). Alternatively, cDNA library can be prepared from a field sample by methods known to a person of ordinary skill, for example, isolating mRNAs and transcribing mRNAs into cDNAs by reverse transcriptase (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). Methods for constructing a genomic library from a donor organism is known to one of ordinary skill in the art and (see, Old, et al., Principles of Gene Manipulation (5th ed.), Blackwell Science, Oxford, U.K. (1994)). Genomic DNAs represented in BAC (bacterial artificial chromosome), YAC (yeast artificial chromosome), or TAC (transformation-competent artificial chromosome, Lin et al., Proc. Natl. Acad. Sci. USA, 96:6535-6540 (1999)) libraries can be obtained from public or private repositories. Alternatively, a pool of genes, which are overexpressed in a tumor cell line compared with a normal cell line, can be prepared or obtained from public or private repositories. Zhang et al (Science, 276: 1268-1272 (1997)) report that using a method of serial analysis of gene expression (SAGE) (Velculescu et al, Cell, 88:243 (1997)), 500 transcripts that were expressed at significantly different levels in normal and neoplastic cells were identified. The transfection of DNAs that overexpresses in a tumor cell line into a host may cause changes in the host, thus a pool of such DNAs is another source for DNA inserts for this invention. The BAC/YAC/TAC DNAs, DNAs or cDNAs can be mechanically size-fractionated or digested by an enzyme to smaller fragments. The fragments are ligated to adapters with cohesive ends, and shotgun-cloned into the episomal non-transforming non-viral vectors. Alternatively, the fragments can be blunt-end ligated into the episomal non-transforming non-viral vectors. Recombinant the episomal non-transforming non-viral vectors containing an insert or a nucleic acid sequence derived from the cDNA library or genomic DNA library is then constructed using conventional techniques. The episomal non-transforming non-viral vectors produced comprise the nucleic acid insert derived from the donor organism. The insert is transcribed as RNA in the host; the RNA is capable of regulating the expression of a phenotypic trait by a positive sense or antisense mechanism. The nucleic acid sequence may also regulate the expression of more than one phenotypic trait. Nucleic acid sequences from Monera, Protista, Fungi, Plantae and Animalia may be used to assemble the DNA libraries. This method may thus be used to discover useful dominant gene phenotypes from DNA libraries through the gene expression in a host.

cDNA insert libraries can be enriched by excluding certain sequences by removing cDNA homologous to cDNA insert libraries prepared from other donor organisms, cells, tissues, organs, or the like through subtractive hybridization. Genomic libraries can also be enriched in this manner. In another aspect of the invention, the genomic or cDNA inserts can be prepared from a single open reading frame or a single gene, a few genes, a group of genes, or any nucleic acid that is less than the entire genome of a donor organism. This subgenomic nucleic acid can be preselected, e.g. the individual genes in the group of genes can each be preselected. The preselected nucleic acid can be known or unknown at the time of selection. A single cDNA insert can be digested with one or more restriction enzymes to obtain a library, or a cDNA can be subjected to nested deletions, from either end of the cDNA, to produce two libraries of cDNA of differing lengths. In another aspect of the invention, the inserts can be pooled together from different donor organisms, either partial of full genomic or cDNA libraries.

### Vectors of the Present Invention

The vectors to be used in the present invention can be a compacted nucleic acid delivered to cells or an episomal vector that contains a papovavirus origin of replication and a papovavirus large T antigen mutant form or both.

The host can be introduced with a vector by conventional techniques. For example, if the host is a cell line, the can be introduced by electroporation, PEG mediation, lipofectin mediation or calcium phosphate precipitation. If the host is a mouse, vector can be introduced by intramuscular or intravenous injection, intranasal passages, or passages through any other suitable membrane. The vector can also be introduced into the host using the following method(s):

### I. Compacted Nucleic Acids and Their Delivery to the Host.

### The Target Cell

The target cells may belong to tissues (including organs) of the organism, including cells belonging to (in the case of an animal) its nervous system (e.g., the brain, spinal cord and peripheral nervous cells), the circulatory system (e.g., the heart, vascular tissue and red and white blood cells), the digestive system (e.g., the stomach and intestines), the respiratory system (e.g., the nose and the lungs), the reproductive system, the endocrine system (the liver, spleen, thyroids, parathyroids), the skin, the muscles, or the connective tissue.

Alternatively, the cells may be cancer cells derived from any organ or tissue of the target organism, or cells of a parasite or pathogen infecting the organism, or virally infected cells of the organism.

The present method for introducing one or more vectors into a host has the following major advantages: (i) ease of preparation of the DNA complex; (ii) ability to target genes to specific tissues; (iii) prolonged expression of the gene in the target organ or tissue (such as the liver); (iv) relative safety of the complex, since it is devoid of infectious viral DNA; and (v) episomal maintenance of the introduced gene.

### TARGETING

### A. Generally

“Targeting” is the administration of the compacted nucleic acid in such a manner that it enters the target cells in amounts effective to achieve the clinical purpose. In this regard, it should be noted that DNA and RNA are capable of replication in the nucleus of the target cell, and in consequence the ultimate level of the nucleic acid in the cell may increase after uptake. Moreover, if the clinical effect is mediated by a protein expressed by the nucleic acid, it should be noted that the nucleic acid acts as a template, and thus high levels of protein expression can be achieved even if the number of copies of the nucleic acid in the cell is low. Nonetheless, it is desirable to compact high concentrations of DNA to increase the number of target cells which take up the DNA and the number of DNA molecules taken up by each cell.

The route and site of administration may be chosen to enhance targeting. For example, to target muscle cells, intramuscular injection into the muscles of interest would be a logical choice. Lung cells might be targeted by administering the compacted DNA in aerosol form. The vascular endothelial cells could be targeted by coating a balloon catheter with the compacted DNA and mechanically introducing the DNA.

In some instances, the nucleic acid binding moiety, which maintains the nucleic acid in the compacted state, may also serve as a targeting agent. Polymers of positively charged amino acids are known to act as nuclear localization signals (NLS) in many nuclear proteins. In some embodiments, targeting may be improved if a target cell binding moiety is employed.

### B. Use Of A Target Binding Moiety (TBM)

If a TBM is used, it must bind specifically to an accessible structure (the “receptor”) of the intended target cells. It is not necessary that it be absolutely specific for those cells, however, it must be sufficiently specific for the conjugate to be therapeutically effective. Preferably, its cross-reactivity with other cells is less than 10%, more preferably less than 5%.

There is no absolute minimum affinity which the TBM must have for an accessible structure of the target cell; however, the higher the affinity, the better. Preferably, the affinity is at least 10³ liters/mole, more preferably, at least 10⁶ liters/mole.

The TBM may be an antibody (or a specifically binding fragment of an antibody, such as a Fab, Fab, V_{M}, V_{L}, or CDR) which binds specifically to an epitope on the surface of the target cell. Methods for raising antibodies against cells, cell membranes, or isolated cell surface antigens are known in the art. Furthermore, the TBM may comprise a single-chain Fv which binds specifically to an epitope on the surface of the target cell. The single-chain Fv may comprise a fusion protein with a NABM or a therapeutic protein sequence (e.g, an enzyme, cytokine, protein antibiotic, etc.).

The TBM may be a lectin, for which there is a cognate carbohydrate structure on the cell surface.

The target binding moiety may be a ligand which is specifically bound by a receptor carried by the target cells.

One class of ligands of interest are carbohydrates, especially mono- and oligosaccharides. Suitable ligands include galactose, lactose and mannose.

Another class of ligands of interest are peptides (which here includes proteins), such as insulin, epidermal growth factor(s), tumor necrosis factor, prolactin, chorionic gonadotropin, FSH, LH, glucagon, lactoferrin, transferrin, apolipoprotein E, gp120 and albumin.

The following table lists preferred target binding moieties for various classes of target cells:

| Target Cells | Target Binding Moiety |
|---|---|
| liver cells | galactose |
| Kupffer cells | mannose |
| macrophages | mannose |
| lung, liver, intestine | Fab fragment vs. polymeric |
| | immunoglobulin receptor (pIg R) |
| adipose tissue | insulin |
| lymphocytes | Fab fragment vs CD4 or gp120 |
| enterocyte | Vitamin B12 |
| muscle | insulin |
| fibroblasts | mannose-6-phosphate |
| nerve cells | Apolipoprotein E |

The target binding moiety may be encompassed with a larger peptide or protein. The present invention provides peptides containing the pentapeptide binding domain for the serpin enzyme complex (SEC) receptor. The present invention further contemplates the production of retroviral particles comprising modified (i.e., chimeric) envelope proteins containing protein sequences comprising a target binding moiety capable of binding to a SEC receptor (or any other desired receptor).

Retrovirus particles bearing these modified envelope proteins may be used to deliver genes of interest to cells expressing the SEC receptor. Retroviral particles bearing chimeric proteins containing peptide ligands and a portion of the envelope (env) protein of retroviruses (e.g., ecotropic Moloney murine leukemia virus or avian retroviruses) have been shown to be capable of binding to cells expressing the cognate receptor (Kasahara et al. (1994) Science 266:1373 and Valsesia-Wittmann et al. (1994) J. Virol. 68:4609).

The use of a target binding moiety is not strictly necessary in the case of direct injection of the NABM/NA condensed complex. The target cell in this case is passively accessible to the NABM/NA condensed complex by the injection of the complex to the vicinity of the target cell.

### C. Liposome-Mediated Gene Transfer

The possibility of detecting gene expression by encapsulating DNA into a liposome (body contained by a lipid bilayer) using various lipid and solvent conditions, and injecting the liposome into animal tissues, has been demonstrated. However, despite the potential of this technique for a variety of biological systems, the DNA used in these experiments has not been modified or compacted to improve its survival in the cell, its uptake into the nucleus or its rate of transcription in the nucleus of the target cells. Thus, these procedures have usually resulted in only transient expression of the gene carried by the liposome.

Cationic lipids have been successfully used to transfer DNA. The cationic component of such lipids can compact DNA in solution. This method has been shown to result in heavily aggregated DNA complexes that, when used for transfecting the DNA in vitro, results in increased efficiency of gene transfer and expression (relative to naked DNA). Although the formation of these complexes can promote gene transfer in vitro, the injection of such complexes in vivo does not result in long lasting and efficient gene transfer.

The condensation procedure of the present invention provide structural features to the DNA/cationic lipid complex that will make it more amenable to prolonged in vivo expression. The combination of such methods could be accomplished by either of two procedures: (1) Formation of condensed DNA complex that is later encapsulated using neutral lipids into liposome bodies, or (2) using the procedure described in this patent, the formation of highly condensed unimolecular DNA complexes upon condensation with cationic lipids could be accomplished. These complexes should provide a higher efficiency of gene transfer into tissues of animals in vivo.

The procedure of the present invention for the condensation of DNA, if coupled to the encapsulation of the resulting compacted DNA into a liposome body, could provide a variety of advantages for transfection into animals:

1. The liposome promotes the passive fusion with the lipid bilayer of the cytoplasmic membrane of mammalian cells in tissues.

2. The condensed DNA could then transfer the genetic information with a higher efficiency through the cell compartments to the nucleus for its expression.

3. Condensed DNA could be protected against degradation inside the cell, thus augmenting the duration of the expression of the newly introduced gene.

4. Possible immunological response to the polycation condensed DNA could be avoided by the encapsulation with the immunologically inert lipid bilayer.

### The Nucleic Acid Binding Moiety

Any substance which binds reversibly to a nucleic acid may serve as the nucleic acid binding moiety (NABM), provided that (1) it binds sufficiently strongly and specifically to the nucleic acid to retain it until the conjugate reaches and enters the target cell, and does not, through its binding, substantially damage or alter the nucleic acid and (2) it reduces the interactions between the nucleic acid and the solvent, and thereby permits condensation to occur.

Preferably, the NABM is a polycation. Its positively charged groups bind ionically to the negatively charged DNA, and the resulting charge neutralization reduces DNA-solvent interactions. A preferred polycation is polylysine. Other potential nucleic acid binding moieties include Arg-Lys mixed polymers, polyarginine, polyornithine, histones, avidin, and protamines.

### The Nucleic Acid

Basic procedures for constructing recombinant DNA and RNA molecules in accordance with the present invention are disclosed by Sambrook, J. et al., In: Molecular Cloning: A Laboratory Manual, Second Edition, Cold Spring Harbor Press, Cold Spring Harbor, N.Y. (1989), which reference is herein incorporated by reference.

The nucleic acid may be a DNA, RNA, or a DNA or RNA derivative such as a derivative resistant to degradation in vivo, as discussed below. Within this specification, references to DNA apply, mutatis mutandis, to other nucleic acids as well, unless clearly forbidden by the context. The nucleic acid may be single or double stranded. It is preferably of 10 to 1,000,000 bases (or base pairs), more preferably 100 to 100,000, and the bases may be same or different. The bases may be the “normal” bases adenine (A), guanine (G), thymidine (T), cytosine (C) and uracil (U), or abnormal bases such as 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, β,D-galactosylqueuosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, β,D-mannosylqueuosine, 5-methoxycarbonylmethyl-2-thiouridine, 5-methoxycarbonylmethyluridine, 5-methoxyuridine, 2-methylthio-N6-isopentenyladenosine, N-((9-β-D-ribofuranosyl-2-methylthiopurine-6-yl)carbamoyl) threonine, N-((9-β-D-ribofuranosylpurine-6-yl)-methylcarbamoyl) threonine, uridine-5-oxyacetic acid-methylester, uridine-5-oxyacetic acid, wybutoxosine, pseudouridine, queuosine, 5-methyl-2-thiouridine, 2-thiocytidine, 5-methyl-2-thiouridine, 2-thiouridine, 4-thiouridine, 5-thiouridine, N-((9-β-D-ribofuranosylpurine-6-yl) carbamoyl)threonine, 2′-O-methyl-5-methyluridine, 52′-O-methyluridine, wybutosine, 3-(3-amino-3-carboxy-propyl) uridine, and the like. The nucleic acid may be prepared by any desired procedure.

In a preferred embodiment, the nucleic acid comprises an expressible gene which is functional in the target cell. For example, the gene may encode coagulation factors, (such as Factor IX), enzymes involved in specific metabolic defects, (such as urea cycle enzymes, especially ornithine transcarbamylase, argininosuccinate synthase, and carbamyl phosphate synthase); receptors, (e.g., LDL receptor); toxins; thymidine kinase to ablate specific cells or tissues; ion channels (e.g., chloride channel of cystic fibrosis); membrane transporters (e.g., glucose transporter); and cytoskeletal proteins, (e.g., dystrophin). The gene may be of synthetic, cDNA or genomic origin, or a combination thereof. The gene may be one that occurs in nature, a non-naturally occurring gene which nonetheless encodes a naturally occurring polypeptide, or a gene which encodes a recognizable mutant of such a polypeptide. It may also encode an mRNA which will be “antisense” to a DNA found or an mRNA normally transcribed in the host cell, but which antisense RNA is not itself translatable into a fIuctional protein.

For the gene to be expressible, the coding sequence must be operably linked to a promoter sequence functional in the target cell. Two DNA sequences (such as a promoter region sequence and a coding sequence) are said to be operably linked if the nature of the linkage between the two DNA sequences does not (1) result in the introduction of a frame-shift mutation in the region sequence to direct the transcription of the desired gene sequence, or (3) interfere with the ability of the gene sequence to be transcribed by the promoter region sequence. A promoter region would be operably linked to a DNA sequence if the promoter were capable of effecting transcription of that DNA sequence. In order to be “operably linked” it is not necessary that two sequences be immediately adjacent to one another. A nucleic acid molecule, such as DNA, is said to be “capable of expressing” a mRNA if it contains nucleotide sequences which contain transcriptional regulatory information and such sequences are “operably linked” to nucleotide sequences which encode the RNA. The precise nature of the regulatory regions needed for gene expression may vary from organism to organism, but in general include a promoter which directs the initiation of RNA transcription. Such regions may include those 5′-non-coding sequences involved with initiation of transcription such as the TATA box.

If desired, the non-coding region 3′ to the gene sequence coding for the desired RNA product may be obtained. This region may be retained for its transcriptional termination regulatory sequences, such as those which provide for termination and polyadenylation. Thus, by retaining the 3′-region naturally contiguous to the coding sequence, the transcriptional termination signals may be provided. Where the transcriptional termination signals are not satisfactorily functional in the expression host cell, then a 3′ region functional in the host cell may be substituted.

The promoter may be an “ubiquitous” promoter active in essentially all cells of the host organism, e.g., for mammals, the beta-actin promoter, or it may be a promoter whose expression is more or less specific to the target cells. Generally speaking, the latter is preferred. A promoter native to a gene which is naturally expressed in the target cell may be used for this purpose, e.g., the PEPCK (phosphoenol pyruvate carboxykinase) promoter for expression in mammalian liver cells. Other suitable promoters include albumin, metallothionein, surfactant, apoe, pyruvate kinase, LDL receptor HMG CoA reductase or any promoter which has been isolated, cloned and shown to have an appropriate pattern of tissue specific expression and regulation by factors (hormones, diet, heavy metals, etc.) required to control the transcription of the gene in the target tissue. In addition, a broad variety of viral promoters can be used; these include MMTV, SV-40 and CMV. An “expression vector” is a vector which (due to the presence of appropriate transcriptional and/or translational control sequences) is capable of expressing a DNA (or cDNA) molecule which has been cloned into the vector and of thereby producing an RNA or protein product. Expression of the cloned sequences occurs when the expression vector is introduced into an appropriate host cell. If a prokaryotic expression vector is employed, then the appropriate host cell would be any prokaryotic cell capable of expressing the cloned sequences. Similarly, when an eukaryotic expression vector is employed, then the appropriate host cell would be any eukaryotic cell capable of expressing the cloned sequences.

In addition to or instead of an expressible gene, the nucleic acid may comprise sequences homologous to genetic material of the target cell, whereby it may insert itself (“integrate”) into the genome by homologous recombination, thereby displacing a coding or control sequence of a gene, or deleting a gene altogether.

In another embodiment, the nucleic acid molecule is “antisense” to a genomic or other DNA sequence of the target organism (including viruses and other pathogens) or to a messenger RNA transcribed in cells of the organisms, which hybridizes sufficiently thereto to inhibit the transcription of the target genomic DNA or the translation of the target messenger RNA. The efficiency of such hybridization is a function of the length and structure of the hybridizing sequences. The longer the sequence and the closer the complementarily to perfection, the stronger the interaction. As the number of base pair mismatches increases, the hybridization efficiency will fall off. Furthermore, the GC content of the packaging sequence DNA or the antisense RNA will also affect the hybridization efficiency due to the additional hydrogen bond present in a GC base pair compared to an AT (or AU) base pair. Thus, a target sequence richer in GC content is preferable as a target.

It is desirable to avoid antisense sequences which would form secondary structure due to intramolecular hybridization, since this would render the antisense nucleic acid less active or inactive for its intended purpose. One of ordinary skill in the art will readily appreciate whether a sequence has a tendency to form a secondary structure. Secondary structures may be avoided by selecting a different target sequence.

An oligonucleotide, between about 15 and about 100 bases in length and complementary to the target sequence may be synthesized from natural mononucleosides or, alternatively, from mononucleosides having substitutions at the non-bridging phosphorous bound oxygens. A preferred analogue is a methylphosphonate analogue of the naturally occurring mononucleosides. More generally, the mononucleoside analogue is any analogue whose use results in oligonucleotides which have the advantages of (a) an improved ability to diffuse through cell membranes and/or (b) resistance to nuclease digestion within the body of a subject (Miller, P. S. et al., Biochemistry 20:1874-1880 (1981)). Such nucleoside analogues are well-known in the art. The nucleic acid molecule may be an analogue of DNA or RNA. The present invention is not limited to use of any particular DNA or RNA analogue, provided it is capable of fulfilling its therapeutic purpose, has adequate resistance to nucleases, and adequate bioavailability and cell take-up. DNA or RNA may be made more resistant to in vivo degradation by enzymes, e.g., nucleases, by modifying internucleoside linkages (e.g., methylphosphonates or phosphorothioates) or by incorporating modified nucleosides (e.g., 2′-O-methylribose or 1′-alpha-anomers). The entire nucleic acid molecule may be formed of such modified linkages, or only certain portions, such as the 5′ and 3′ ends, may be so affected, thereby providing resistance to exonucleases.

Nucleic acid molecules suitable for use in the present invention thus include but are not limited to dideoxyribonucleoside methylphosphonates, see Mill, et al., Biochem., 18:5134-43 (1979), oligodeoxynucleotide phosphorothioates, see Matsukura, et al., Proc. Nat. Acad. Sci. USA, 84:7706-10 (1987), oligodeoxynucleotides covalently linked to an intercalating agent, see Zerial, et al., Nucleic Acids Res., 15:9909-19 (1987), oligodeoxynucleotide conjugated with poly(L-lysine), see Leonetti, et al., Gene, 72:32-33 (1988), and carbamate-linked oligomers assembled from ribose-derived subunits, see Summerton, J., Antisense Nucleic Acids Conference, 37:44 (New York 1989).

### Compaction Of The Nucleic Acid

It is desirable that the complex of the nucleic acid and the nucleic acid binding moiety be compacted to a particle size which is sufficiently small to achieve uptake by receptor mediated endocytosis, passive internalization, receptor-mediated membrane permeabilization, or other applicable mechanisms. Desirably, the complex of the compacted nucleic acid, the target binding moiety, and the nucleic acid binding moiety is small, e.g., less than 100 nm, because the sinusoidal capillary systems of the lung and spleen will trap aggregates of that size, and more preferably less than 80 or 90 nm, as that is the typical internal diameter of coated-pit endocytic vesicles. Since complexes larger than 30 nm maybe susceptible to nonspecific takeup by macrophages in the spleen and liver, the conjugate is preferably also smaller than 30 nm.

In the case of the ASGP receptor of the liver, complexes larger than 15-23 nm are excluded from uptake. This size limitation in vivo for the receptor is probably directly related to the existence of another receptor for galactosylated proteins in the Kupffer cells of the liver. The Kupffer cell receptor is very efficient in taking up and degrading galactosylated molecules of larger size in vivo and thus, would compete for the uptake of the galactosylated DNA complex with the ASGP receptor on the surface of hepatocytes. Most preferably, for liver delivery, the complex is less than 23 nm, more preferably less than 15 nm, still more preferably no more than 12 nm in diameter.

The present invention calls for the complex of the nucleic acid and the nucleic acid-binding carrier to be compacted without causing aggregation or precipitation, and preferably to a condensed state. For the purpose of the present invention, it is helpful to characterize DNA as having one of the following states: normal (uncondensed); condensed; relaxed; uni-aggregated (clusters of unimolecular toroids); multi-aggregated (clusters of multimolecular toroids); and precipitated. These states are defined in terms of their appearance under electron microscopy (see Table 103 of U.S. Pat. No. 5,972,901).

Condensed DNA is in a state in which interaction with the solvent is minimal and therefore the DNA is in the form of isolated spheres or toroids. It is not fibrous to an appreciable degree. Relaxed DNA, typically formed by dissociation of polycation from the DNA, forms fibers. Aggregated DNA forms clumped or multimolecular toroids.

The theoretical size of a unimolecular DNA complex can be calculated using the method described in U.S. Pat. No. 5,972,901. Preferably, the complexes of this invention have a diameter which is less than double the size calculated by one or both of these formulae. Larger complexes are likely to correspond to multimolecularly aggregated DNA.

DNA can be compacted to a condensed state by neutralizing its charge, e.g., by addition of a polycation, or otherwise reducing its interactions with solvent. However, the polycation can cause aggregation or precipitation of the DNA if a chaotropic agent is not employed to prevent it. Compaction therefore can be accomplished by judicious use of both the polycation (to condense the DNA) and (as needed) of a chaotropic agent (to prevent aggregation or precipitation). Preferably, the complex has a unaggregated, unimolecular toroid structure condensed to smaller than 23 nm in diameter; the degree of compaction may be determined by electron microscopy.

The term “unimolecular toroid” indicates that the toroid contains only one nucleic acid molecule; the toroid may contain many carrier (e.g., galactosylated poly-Lys) molecules. A typical ratio is one DNA molecule to about 100 carrier molecules, per “unimolecular” toroid. Alternatively, and perhaps more precisely, this structure may be referred to as a mono-nucleic acid toroid. Unimolecular and multimolecular toroids (the latter each contain more than one DNA molecule) may be distinguished by the different size of each of the complexes when viewed by the electron microscope, indicating the multi- or unimolecular (counting only the DNA molecules) composition of the toroids.

We have also used other techniques to identify structural changes in the DNA upon poly-L-lysine binding. The first of these is the spectrophotometric determination of the turbidity in the solution using the absorbance at 400 nm. Turbidity is primarily an indicator of aggregation. Aggregation is confirmed by a circular dichroism (CD) value greater than 0 at wavelengths from 300 to 340 nm.

The turbidity of a solution containing the DNA complexes is dependent on the initial concentration of salt used for condensation of the complex. Although the mechanisms responsible for the observed differences in the condensation of DNA at initial low and high ionic strength is not clear, we adapted our protocol to appropriately condense DNA, avoiding the formation of turbid solutions.

A more reliable technique for diagnosing the structural transition of DNA-poly-L-lysine complexes in solution is the absorbance of the condensing complex at 260 mn as the concentration of NaCl increases. The uni-aggregated DNA complex in suspension has only 10-30% of the expected absorbance because the particulate matter does not absorb at 260 nm. The addition of NaCl disperses the uni-aggregated DNA complex in suspension which results in the observed steep increase in the absorbance. At this point the solution is clear and there are no visible particulate structures in suspension. At a concentration of NaCl which causes a steep increase in the absorbance at 260 nm, unaggregated, condensed complexes canm be observed by EM; before this critical concentration of NaCl was attained, the DNA complex is aggregated and at higher NaCl concentrations the DNA complex is relaxed. A second transition in absorbance at 260 mn, as a result of the relaxation of the condensed DNA complex that was in suspension, indicates the full solubilization of the DNA complex.

Circular dichroism (CD) can be used to monitor the condensation of DNA. When the spectrum is identical to that of DNA alone, then the DNA complex is assumed to be correctly compacted, i.e., into unimolecular complexes. In another words, the positive spectrum at 220 nm is quantitatively similar to the 220 nm spectrum of DNA alone, and the cross-over (the wavelength at which the spectrum of the complex crosses the 0 point) is essentially identical to that of DNA alone. When the DNA aggregates into multimolecular complexes, the positive spectrum at 270 nm is inverted into a negative spectrum at that wavelength (this is called psi-DNA structure or ψ-DNA).

To compact the nucleic acid, the carrier is added to the nucleic acid solution, whereby the carrier disrupts the nucleic acid: solvent interactions allowing the nucleic acid to condense. Preferably, at least the turbidity of the solution is monitored as the carrier is added, so that a change in state is promptly detected. Once turbidity appears, the state of the DNA may be further analyzed by CD spectroscopy to determine whether the DNA is in the condensed or the aggregated state. (Precipitation should also be detectable with the naked eye.) Preferably, the carrier is added sufficiently slowly to the nucleic acid solution so that precipitation and aggregation are minimized. If precipitation or aggregation occur, a chaotropic salt should be added slowly, and the result again examined by CD spectroscopy. The preferred salt is NaCl. Other chaotropic salts can be used as long as they are tolerated by the animal (or cells) to which they will be administered. Suitable agents include Sodium sulfate (Na₂SO₄), Lithium sulfate (Li₂SO₄), Ammonium sulfate ((NH₄)₂SO₄), Potassium sulfate (K2SO4), Magnesium sulfate (MgSO4), Potassium phosphate (KH2PO4), Sodium phosphate (NaH2PO4), Ammonium phosphate (NH4H2PO4), Magnesium phosphate (MgHPO4), Magnesium chloride (MgCl2), Lithium chloride (LiCl), Sodium chloride (NaCl), Potassium chloride (KC1), Cesium chloride (CaCl), Ammonium acetate, Potassium acetate, Sodium acetate, Sodium fluoride (NaF), Potassium fluoride (KF), Tetramethyl ammonium chloride (TMA-Cl), Tetrabutylammonium chloride (TBA-Cl), Trimethylammonium chloride (TEA-Cl), and Methyltriethylammonium chloride (MTEA-Cl).

Many variables affect condensation of DNA in vitro and the functional relevance of these parameters for efficient delivery of DNA complexes into animals by receptor-mediated endocytosis. There is a strong correlation between the ionic strength at which the condensed DNA-poly-L-lysine complex remains stable in solution and the concentration of DNA. These experiments were performed using a 4.5 kb plasmid containing the promoter from the gene for PEPCK linked to the structural gene for hFIX, using a ratio of DNA to poly-L-lysine that resulted in a 1 to 1 ratio of negative to positive charges in solution. The variation in the final concentration of NaCl necessary to solubilize the particles is a logarithmic function of DNA concentration, in which the condensation of highly concentrated DNA-poly-L-lysine complexes occurs with only a slight increase in ionic strength. This physical characteristic of DNA condensation has clear advantages for the delivery of the DNA particles to tissues of adult animals in vivo since it has little effect on the ionic load in the animal's blood.

The linear fit of the data using the least square method is described by the following function:

log₁₀(NaCl, mM)=b0*(DNA, μM Phosphate)+b1r2=0.97

where b0=2.52×10E-3, b1=0.577

Variations in the function described by the above equation are observed when different DNA plasmids and different DNA preparations are used during the condensation process. These differences are probably related to the variation in the affinity of poly-L-lysine for DNA of different sources and compositions. For maximum binding affinity generally DNA precipitated twice with sodium acetate and 2.5 volumes of −40° C. ethanol is used. There is no apparent difference in binding affinity of poly-L-lysine for DNA of different forms (i.e., supercoiled, nicked and linear) and for DNA extracted using anionic exchange chromatography or cesium chloride gradient centrifugation. This may indicate the presence of a contaminant in the DNA preparations from different sources which has poly-L-lysine binding activity, that is eliminated by sequential DNA precipitation.

The length of the poly-L-lysine affects the concentration of NaCl necessary for the effective condensation of DNA (see FIG. 19 of U.S. Pat. No. 5,972,901). There is a correlation between the length of the poly-L-lysine and the necessary concentration of NaCl needed for the condensation of the DNA complex in solution. This correlation is a linear function of poly-L-lysine length up to a size of 150 lysine residues, after which the function reaches saturation and there is no increase in the concentration of NaCl needed for the condensation of DNA with longer poly-L-lysine. This is consistent with a cooperative binding between the poly-L-lysine and the DNA phosphate backbone. Thus, by reducing the length of the poly-L-lysine molecules used to condensed the DNA the solution of DNA complex injected into a host will be less hypertonic. It is also important to consider the dilution of the DNA complex in the blood of the host to evaluate the functional significance of these changes in ionic strength on the efficiency of this method for gene therapy. Rats injected with DNA complexes containing longer range of poly-L-lysine lengths and rabbits with the shorter range of sizes of poly-L-lysine produce positive and persistent expression of the transfected genes in both cases.

The preferred minimum initial salt concentration is dependent on the compaction activity of the carrier and the chaotropic activity of the salt. If the NABM were (Lys)₈, or (Lys)₂₇, the initial NaCl concentration could be zero. With longer polyLys chains, however, in the absence of NaCl, precipitation would be immediate. With (Lys)₅₀, the initial NaCl concentration is preferably be at least about 300 mM. Nonetheless, if the TBM is a protein that affects the condensation, the initial salt concentration could be as low as zero.

The carrier may be added continuously, or in small discrete steps. One may begin with a higher flow rate, or larger aliquots, and reduce the flow rate or aliquot size as the desired endpoint of the reaction is neared. Typically 0.1 to 10% of the carrier solution is added at a time to the DNA solution. Each addition is preferably made every 2 seconds to 2 minutes, with constant vortexing. However, longer settlement times may be allowed.

In one embodiment, a nucleic acid, contained in a salt solution, which is preferably at least 0.5M, but less than 1.5M NaCl, is mixed with poly-L-lysine (109 lysines) containing the covalently linked target cell binding moiety (for example, galactose), which is contained in a solution of NaCl at the same concentration (e.g., 0.5 to 1.5M NaCl). Preferably, the molar ratio of nucleic acid phosphate group to positively charged group of the DNA binding moiety is in the range of 4:1 to 1:4, and more preferably is about 1.5:1.

### The Conjugation

In the embodiments relying on a target-binding carrier molecule, the nucleic acid binding moiety will be conjugated, covalently or noncovalently, directly or indirectly, to the target cell binding moiety. The conjugation may be performed after, or, more usually before, the loading of the nucleic acid binding moiety with the nucleic acid of interest. Either way, the conjugation should not substantially interfere with the binding of the nucleic acid to the nucleic acid binding moiety, or, for that matter, with the ability of the target cell binding moiety to bind to the target cell.

### Pharmaceutical Compositions And Methods

The compacted nucleic acid, optionally conjugated with a TBM, may be admixed with a pharmaceutically acceptable excipient (i.e., carrier) for administration to a human or other animal subject. It will be appreciated that it is possible for a DNA solution to contain both condensed DNA and relaxed DNA. The compositions of this invention preferably are sufficiently rich in condensed complexes so that the absorbance at 260 nm is less than 50% that of naked DNA of equal concentration. Typically, condensed DNA usually has an absorbance of 20-30%, and relaxed DNA, 80-100%, that of naked DNA.

The administration may be by any suitable route of administration. The dosage form must be appropriate for that route. Suitable routes of administration and dosage forms include intravascular (injectable solution), subcutaneous (injectable solution, slow-release implant), topical (ointment, salve, cream), and oral (solution, tablet, capsule). With some routes of administration, the dosage form must be formulated to protect the conjugate from degradation, e.g., by inclusion of a protective coating or of a nuclease inhibitor.

The dosage may be determined by systematic testing of alternative doses, as is conventional in the art. Rats (200-300 g) tolerate as much as 600 μg doses of the DNA complex of Example 1 of U.S. Pat. No. 5,972,901 without any apparent ill effects on growth or health. Mice (25 g) have been injected with 150 μg of that DNA complex without any apparent problem. In humans, a typical trial dose would be 60-120 mg of DNA; if this dose is too low to be effective or so high as to be toxic, it may be increased, or decreased, respectively, in a systematic manner, until a suitable dose is identified. For short life span cells, e.g., macrophages, a typical dosing schedule might be one dose every two weeks. For long life span cells, e.g., hepatocytes, one dose every two months might be preferable.

Adjuvants may be used to decrease the size of the DNA complex (e.g., 2-10 mM MgCl), to increase its stability (e.g., sucrose, dextrose, glycerol), or to improve delivery efficiency (e.g., lysosomotropic agents such as chloroquine and monensine). The complexes may be enclosed in a liposome to protect them and to facilitate their entry into the target cell (by fusion of the liposome with the cell membrane).

### II. Episomal Vector That Contains a Papovavirus Origin of Replication and a Papovavirus Large T Antigen Mutant Form.

A suitable vector exemplified for use in the invention is an episomal vector that efficiently replicates in transformed transitional epithelial cells (e.g., HT-1376 bladder carcinoma cell line). The vector (such as pRP-cneoX cited in U.S. Pat. No. 5,624,820) contains a marker gene under control of the SV40 early promoter and a 3.2 kb segment of BKV which includes the BKV origin of replication and the BKV large T antigen under control of the BKV early promoter. Whereas the EBV episomal element was not active in HT-1376 transient transfectants, BKV episomes replicated extrachromosomally in these cells. More importantly, BKV episomes can replicate efficiently in HT-1376 cells without any apparent cellular toxicity, resulting in a high copy number of the episome in stable transfectants.

The copy number of BKV episome pRP-cneoX in HT-1376 cells stably transfected with this construct is approximately 150 copies per cell. This copy number compares to approximately 10-50 copies of EBV-derived episomes in lymphoblastoid cell lines and 10-80 copies of bovine papilloma virus-derived episomes in murine C127 cells (Sarver, et al., 1981, Mol. Cell. Biol. 1:486-96; DiMaio, et al., 1982, Proc. Natl. Acad. Sci. USA 97:4030-34; Yates, et al., 1985, Nature 313:812-15). The high copy number of pRP-cneoX in HT-1376 transfectants is likely responsible for the efficient vertical transfer of pRP-cneoX to the progeny of these HT-1376 transfectants over multiple generations. The soft agar cloning efficiencies of HT-1376 cells transfected with either integrating vector pSV2NEO or pRP-cneoX, and plated in the presence or absence of G418, are essentially identical. This indicates that episomal transfer of the neomycin resistance gene to daughter cells is as efficient as when this gene is integrated into HT-1376 genomic DNA. The probability that a given daughter cell does not contain at least one copy of the episome is very low assuming random partitioning of the large number of plasmid copies during cellular division.

The BKV episomal expression vector can produce very high levels of transcription of a transfected gene in HT-1376 cells. There is approximately a 20-fold increase in the steady-state level of neomycin resistance gene expression in pRP-cneoX transfectants compared to transfectants which have 5 integrated copies of pSV2NEO. Since the neomycin resistance gene is transcriptionally regulated by the SV40 early promoter in both constructs, this demonstrates that BKV episomal vectors can produce significantly higher levels of expression of a transfected gene that plasmid vectors that must integrate into the host cell genome to produce stable transfectants. This difference is presumably due in part to the higher copy number of pRP-cneoX (150 copies) compared to pSV2NEO (5 copies) in HT-1376 transfectants.

### Comparison of Episomal Vectors

BKV-derived episomes have several properties that are distinct from EBV, BPV, and SV40-derived episomes. Despite the significant amino acid homology between the large T antigens from BKV and SV40 (Mann, et al., 1984, Virol., 138: 379-385), BKV episomes can yield stable, viable transfectants whereas SV40-based episomes replicate to such a high copy number that cell death typically ensues (Tsui, et al., 1982, Cell 30:499-508; Roberts, et al., 1986, Cell 52:397-404). This result may be due, in part, to differences in the level of T antigen present in these transfectants, characteristics of the DNA origins from these viruses, or presence of cis-regulatory sequences in the BKV episome that regulate DNA replication, as has been described in composite SV40-BPV-derived episomes (Roberts, et al., 1986; Hambor, et al., 1988, Proc. Natl. Acad. Sci. USA, 85: 4010-14).

Significantly, BKV episomes appear to replicate once per cell cycle in stable transfectants, because the pRP-cneoX copy number reaches a stable plateau of approximately 150 copies per cell. Stable copy number is also characteristic of EBV and BPV-derived episomes, which can similarly yield viable, stable transfectants, albeit at lower copy number. In contrast to EBV-derived episomes (Yates, et al., 1984, Proc. Natl. Acad. Sci. USA 81:3806-10; Hambor, et al., 1988), however, the copy number of BKV episomes is maintained at unreduced levels after 2 months of growth in the absence of selection pressure. pRP-cneoX copy number fluctuates during the time course of G418 withdrawal. This represents a dynamic interplay between factors predisposed to maintain the presence of episomes (such as efficient episomal replication during the cell cycle and potential growth advantages present in cells expressing BKV large T antigen) and factors that may reduce episomal copy number (such as unequal partitioning of the episome during cell division, or destruction by cellular nucleases). Comparable to BKV episomes, BPV episomes can also be maintained at stable copy numbers in unselected, transformed C127 transfectants (Sarver, et al., 1981; DiMaio, et al., 1982). However, the higher copy number of BKV episomes in unselected transfectants is an advantage in strategies to utilize these episomes for gene therapy.

### I. Definitions

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., an intron-free coding sequence (cDNA) where the genomic coding sequence contains introns, or synthetic sequences having codons different than the native gene). Allelic variations or naturally occurring mutational events do not give rise to a heterologous region of DNA as defined herein.

A DNA “coding sequence” is a DNA sequence which is transcribed and translated into a polypeptide in vivo when placed under the control of appropriate regulatory sequences. A polyadenylation signal and transcription termination sequence will usually be located 3′ to the coding sequence. A “promoter” is a DNA regulatory region capable of binding RNA polymerase in a cell and initiating transcription of a downstream (3′ direction) coding sequence. A coding sequence under the control of a promoter in a cell is transcribed by RNA polymerase after the polymerase binds the promoter, the coding sequence being transcribed into mRNA which is then in turn translated into the protein encoded by the coding sequence.

“Transfection” of a cell occurs when exogenous DNA has been introduced inside the cell membrane. “Transformation” occurs when a cell population from primary cells or a cell line that only undergoes a finite number of divisions becomes immortalized, or when an immortal cell line acquires additional tumorigenic properties. Transformation can be detected by, for example, the ability of the transformed cell to form clones in soft agar or to form tumors in nude or SCID mice.

A “clone” is a population of cells derived from a single cell or common ancestor by mitosis.

A “replicon” is any genetic element (e.g., plasmid, chromosome, virus) that functions as an autonomous unit of DNA replication in vivo.

A “vector” is a replicon, such as plasmid, phage or cosmid, to which another DNA segment (a heterologous segment) may be attached so as to bring about the replication of the attached segment.

An “episome” is a low molecular weight DNA molecule that resides in a cell separated from the cell's chromosome(s). Episomes replicate independently of mitotic replication of the chromosomes, being transmitted to daughter cells as part of the random reassortment of cellular contents during cell division. “Copy number” is the number of duplicate DNA molecules existing in an individual cell as episomes or is the number of duplicate sequences in the genome. Bacterial episomes are usually called plasmids.

“Foreign genes” are genes that are not found in the genome of the individual host cell. Foreign genes may be from the same species as the host or from different species. Where this invention describes transfection of a cell using DNA containing a foreign gene with the intent that the foreign gene will be expressed in the cell, the DNA will, of course, contain any control sequences necessary for expression of the foreign gene in the required orientation for expression.

### II. Description of the Vector

### A. Papovaviruses

Papovaviruses are DNA viruses with double-stranded, covalently closed, circular genomes of approximately 5000 bp and icosahedral capsids containing three viral proteins. The papovaviruses infect a variety of hosts, including humans (BK virus and JC virus), monkeys (simian vacuolating virus (SV40) and lymphotropic papovavirus), baboon (simian agent 12), mouse (polyoma virus and K virus), hamster (hamster papovavirus), rabbit (rabbit kidney vacuolating virus), and budgerigar (budgerigar fledgling disease virus). These viruses have been judged to be related based on nucleotide sequence comparisons.

The viral genome is divided into early and late transcription regions, and contains a single origin of replication. Transcription begins from promoters near the origin of replication and proceeds bidirectionally—one direction for early transcripts and the other direction for late transcripts. The late transcriptional region encodes coat proteins (VP1, VP2, and VP3). The early transcriptional region encodes the T antigens, particularly the large T antigen which functions in viral DNA replication. The large T antigen also down-regulates early transcription by binding to viral DNA near the early promoter, activates cellular genes involved in DNA synthesis, and transforms primary cells in tissue culture.

Viral DNA replicates in the nucleus as “minichromosomes,” but viral DNA can replicate many times in a single cellular S phase. Viral DNA replication is initiated by the large T antigen, independently of its stimulation of cellular DNA synthesis. The large T antigen binds to viral DNA in the neighborhood of the origin of replication and unwinds the DNA helix, which is required for viral DNA replication. New viral DNA is then synthesized by cellular enzymes.

### B. Episomal Amplification Cassette

To provide enhanced expression in gene therapy applications, episomal vectors must replicate extrachromosomally without transforming the transfected cell. This invention provides a replication cassette for such containing the essential elements of papovavirus replication. The replication cassette (or episomal amplification cassette) contains (1) a papovavirus origin of DNA replication (ORI); (2) a replication-competent, transformation-negative mutant form of the papovavirus large T antigen; and, (3) a promoter to drive expression of the mutant T antigen. When the replication cassette of this invention is coupled with other DNA sequences in a circular DNA molecule, the DNA molecule will be replicated episomally by mammalian cells after transfection.

The initial BKV episomal vectors reported by Milanesi, et at. (1984), contained a 3.2 kb fragment of BKV including the origin of DNA replication and the BKV large T antigen transcriptionally regulated by the BKV early promoter. The BKV expression system may be modified according to this invention, so that it does not induce soft agar growth in nontumorigenic cells, yet retains the ability to replicate extrachromosomally. The components of the replication cassette will be selected according to the following criteria and assembled as described below.

### 1. Origin of Replication

The origin of replication in the replication cassette is selected from ORI sequences of one of the papovaviruses. DNA replication initiated at these loci is sensitive to control by the large T antigen of the same virus, and to a similar or lesser extent by large T antigen of other papovaviruses.

In the presence of a compatible large T antigen, the papovavirus origin will drive episomal replication. The origin/large T antigen combination should be tested to determine whether they drive replication of the episome. One simple test for replication competency is to transfect a population of cells which express the large T antigen mutant proposed for the replication cassette with a vector containing the proposed origin of replication and then monitor the transfected cells for synthesis of episomal DNA by Southern blot.

Particularly preferred is the BKV origin, which has been demonstrated to drive episomal replication with either BKV large T antigen (BK-T) or SV-T. Other preferred origins are those that drive replication in primates, including SV40, JC virus, lymphotropic papovavirus, and simian agent 12. Any papovavirus origin of replication that can be shown to drive episomal replication in human cells will be suitable for the replication cassettes of this invention.

The BKV replicon is active in the HT-1376 bladder carcinoma cell line, whereas the Epstein-Barr virus (EBV) replicon is not functional in these cells. BKV has a trophism for human uroepithelial cells (Arthur, et al., 1986, N. Engl. J Med., 315:230-234), and an episomal vector derived from BKV will replicate efficiently in human bladder carcinoma cell lines. Hybrid SV40/BK virus-derived episomes replicated extrachromosomally in the nontumorigenic 5637 bladder cell line. This suggests that the tissue tropism of viruses from which episomal constructs are derived may predict the cell type in which episomal constructs are active.

### 2. Large T Antigen Mutants

The replication activity of BKV episomes is dependent on expression of the BK-T. BK-T has a 75% amino acid homology to the SV40 large T antigen (SV-T) (Yang, et al., 1979), a protein having well-described immortalization and tumorigenic properties (Shin, et al., 1975, Proc. Natl. Acad. Sci. USA, 72:4435-39; Christian, et al., 1987, Cancer Res., 47:6066-73; Michalovitz, et al., 1987, J. Virol., 61:2648-54; Hanahan, et al., 1989, Science, 246:1265-75; DeCaprio, et al., 1988, Cell, 54:275-83; Chen, et al., 1990, J. Virol., 64:3350-57; Chen, et al., 1992, Oncogene, 7:1167-75). Similar to SV-T, BK-T can bind to and thereby inactivate wild-type p53 and retinoblastoma (RB) tumor suppressor gene products (Mann, et al., 1984, Virol. 138:379-85; Dyson, et al., 1990, J. Virol. 64:1353-56), the primary proposed mechanism by which these T antigens induce tumorigenic properties (DeCaprio, et al., 1988, Cell 54:275-83; Chen, et al., 1992). Transgenic mice expressing BK-T develop renal carcinomas and thymoproliferative disorders (Dalrymple, et al., 1990, J. Virol., 64:1182-91), and BK-T can transform NIH 3T3 cells and baby rat kidney cells (Nakshatri, et al., 1988, J. Virol., 62:4613-21). It is therefore possible that BKV episomal vectors containing wild-type BK-T could confer tumorigenic properties to some nontumorigenic cell lines, making such an episomal vector unsuitable for use in gene therapy, because the vector may be able to confer soft agar growth on cells in culture or induce neoplastic transformation in vivo.

However, the significant homology between SV-T and BK-T led to a specific strategy to solve this problem. SV-T can bind to the BKV origin of replication in vitro and can stimulate the replication of a plasmid containing the BKV origin of replication in COS cells (Ryder, et al., 1983, Virol., 129: 239-45; Deyerie, et al., 1989, J. Virol., 63:356-65). Therefore, replication-competent SV-T mutants having suppressed transformation properties were examined as substitutes for BK-T to promote replication of BKV episomes without transformation.

### Replication-Competent, Transformation-Negative SV-T Mutants

The domain of SV-T which binds the SV40 DNA origin is separate and distinct from the RB and p53 binding domains. Three replication competent, transformation negative SV-T mutants are exemplified. The first SV-T mutant is 107-T (also referred to as K1, Kalderon, et al., 1984, Virol., 139:109-37), which is replication competent yet nontumorigenic in several cell types (DeCaprio, et al., 1988; Chen, et al., 1990; Chen, et al., 1992; Kalderon, et al., 1984, Virol. 139:109-37; Cherington, et al., 1988, Mol. Cell Biol., 8:1380-84). 107-T differs from wild-type SV-T in a single base pair resulting in substitution of lysine for glutamic acid in codon 107. Codon 107 is in the RB binding domain of SV-T, and the inability of 107-T to bind RB most likely accounts for its nontumorigenic properties. The DNA binding region of 107-T is intact, however, and as shown below, we have determined that 107-T can drive replication of a test plasmid containing the SV40 DNA origin.

The second mutant is 402-T, which has a substitution of glutamic acid for asparagine in codon 402 (Lin, et al., 1991, J. Virol., 65:2066-72). The 402-T point mutation is in the p53 binding domain of SV-T, and 402-T fails to bind wild-type p53, although it appears to bind RB, and can also drive replication of the SV40 DNA origin. 402-T is nontransforming in human diploid fibroblast lines D.551 and WI-38 (Lin, et al., 1991, J. Virol., 65:6447-53).

A novel SV-T mutant has been constructed which contains both point mutations found in 107-T and 402-T (107/402-T). This SV-T mutant will not bind either p53 or RB, and will have very low potential to confer tumorigenic properties.

Integrating vectors encoding these three different SV-T mutants which have differing abilities to bind to wild-type p53 and RB have been prepared, and these SV-T mutant vectors have been transfected into nontumorigenic bladder cell lines. Single cell clones have been characterized which express the mutant SV-T molecules, yet remain nontumorigenic, and some of these clones have been shown to drive replication of plasmids containing SV40 DNA origins. This strategy has therefore been successful in modifying a papovavirus large T antigen for use in episomal vectors carrying an SV40 replicon for efficient expression of foreign genes in nontumorigenic cells.

The large T antigen mutants encoded by replication cassettes of this invention must be replication-competent and transformation-negative, that is they must induce DNA replication and not transform the host cell. Trans-activation of DNA replication can be tested using Southern blot analysis of Hirt supernatant or total cellular DNA extracted from transient episomal transfectants, as described above.

The transforming activity of the mutant large T antigen can be tested directly (see, e.g., Nakshatri, et al. 1988) or cells transfected with an expression vector expressing the mutant T antigen can be tested for soft agar cloning activity or growth in nude or SCID mice to determine whether the mutant T antigen is transformation-negative. Alternatively, mutants may be selected based on negative binding studies with wild-type p53 and wild-type RB. One suitable assay measures binding by generating in vitro translated mutant large T antigen protein and mixing it with authentic wild-type p53 or RB (e.g. in vitro translated or baculovirus produced) before immunoprecipitation with antisera to p53 or RB, respectively, to immunoprecipitate these proteins and any T antigen proteins complexed to them. Western blots of the immunoprecipitate may be developed with antisera to large T antigen, which will detect mutant T antigens that are positive for binding.

An alternative procedure is transfecting a population of mammalian cells expressing wild-type p53 and RB (preferably from a human cell line) with an expression vector so that the cells express the large T mutant (as detected by, e.g., binding to antisera for T antigen). The cells are then lysed and the lysate treated with antisera to p53 or RB. The immunoprecipitate is treated as before. This latter assay has some potential for false-negatives if, for instance, the amount of mutant T antigen expressed is significantly different from the amount of p53 or RB present, or if there are subtle mutations in the p53 or RB expressed by the test cell, but it more closely approximates the in vivo conditions.

A particularly preferred mutant large T antigen is the SV-T mutant 107/402-T described above. Other mutants of SV-T and other papovavirus large T antigens have been described in the literature, and additional mutants can be generated by well-known recombinant DNA techniques. These mutants will be suitable for the replication cassette of this invention, so long as they are replication-competent and transformation-negative as determined by the above tests.

### 3. Promoters

In general, replication-competent, transformation-negative papovavirus large T antigen will be transcriptionally regulated by either heterologous or homologous promoters. The heterologous promoters are usually promoters which are active in mammalian cells, such as mammalian promoters and mammalian viral promoters. Where the episomal amplification cassette is part of an episomal expression vector for gene therapy application, the promoter will, of course, be chosen to be active in the cell which is the target for expression of the foreign gene.

Some heterologous promoters, such as CMV immediate early promoter-enhancer, are not down-regulated by T antigen, thereby maximizing T antigen expression and consequently, episomal replication. This may be particularly advantageous in transient transfection strategies for gene therapy applications in which high level gene expression is desirable. Alternatively, use of homologous papovaviruses promoters, which are down regulated by T antigen, may constrain runaway episomal replication, thereby achieving controlled, stable expression. Such a promoter/T antigen/origin combination will provide high copy number, stable episomes in transfected cells.

Alternatively, the promoter controlling expression of the mutant large T antigen may be selected to regulate episomal replication. For example an inducible promoter (such as the metallothionen promoter) may be used, and replication of the episome will be amplified in the presence of the inducer. Alternatively, a promoter for a developmentally-controlled or tissue-specific gene (e.g., the breast specific promoter for the whey acidic protein gene, hoeneberger, et al., 1988, EMBO J., 7:169-75) may be used to limit the amplification of the episome copy number to certain cell types where that promoter is active. In gene therapy using an episome which carries a foreign gene whose expression level is proportional to copy number, selection of the promoter controlling T antigen expression provides a measure of therapeutic control of expression.

### 4. Vectors for Insertion of Cassettes

The vectors into which the replication cassette of this invention may be inserted may be any vector that will carry the cassette, and any associated foreign genes, into mammalian cells in which the particular papovavirus origin and large T antigen will drive replication of the vector. The vector, of course, will not contain any sequences that prevent replication from the papovavirus origin of replication in mammalian cells or prevent expression of any foreign gene inserted into the vector for gene therapy applications. Suitable vectors include bacterial plasmids, which are useful as shuttle vectors to produce large quantities of the vector containing the replication cassette in bacterial culture for subsequent use in transfection of mammalian cells. Other suitable vectors include well-known mammalian vectors, usually of vital origin, which are known to transfect mammalian cells, and are non-pathogenic, or of limited pathogenicity, including defective or mutant viruses (see, e.g., Hock, et al. 1986, Nature, 320:275-77; Sorrentino, et al. 1992, Science, 257:99-103; Bayle, et at. 1993, Human Gene Therapy, 4:161-70; Le Gal La Salle, et al. 1993, Science, 259:988-90; Quantin, et al. 1992, Proc. Natl. Acad. Sci. USA, 89:2581-84; Rosenfeld, et al. 1992, Cell, 68:143-55). Where the vector is a mammalian virus, it is of course important that insertion of foreign genes into the vital genome does not destroy viral infectivity. Selection of a particular vector will take into account the particular mammal and the particular cell type in which episomal amplification is desired, and the skilled worker can readily select suitable vectors from among many available in art. (See, e.g., Sambrook, et al., 1989, “Molecular Cloning: A Laboratory Manual”; Miller, et al., 1989, BioTechnqiues, 7:980-90; Salmons, et al., 1993, Human Gene Therapy, 4:129-41; Stratford-Perricaudet, et al., 1991, in “Human Gene Transfer,” Cohen-Haguenauer, et al., eds., John Libbery Eurotest Ltd. 219:51-61).

### III. Method of Constructing the Vector

### A. Sources of Component DNA Sequences

The DNA sequences of various papovaviruses are described in the literature, including the DNA sequences encoding the origin of replication, the early promoter, and the large T antigen. (See, e.g., (for SV40) Subramanian, et al. 1977, J. Biol. Chem., 252:355-67; Reddy, et al. 1978, Science, 200:494-502; Fiers, et al. 1978, Nature, 273:113-20; Van Heuverswyn, et al. 1978, Eur. J Biochem., 100:51-60; (for BKV) Yang, et al. 1979, Science, 206:456-61; Deyerle, et al. 1989, J. Virol., 63:356-65; (for hamster papovavirus) Delmas, et al. 1985, EMBO J., 4:1279-86; (for JC virus) Frisque, et al. 1984, J. Virol., 51:458-69; (for polyoma) Zhu, et al. 1984, J. Virol., 51:170-80.) Clones containing many of the sequences are contained in various mammalian vectors available from commercial suppliers, such as Stratagene, Gibco-BRL Life Technologies, United States Biochemicals, and Promega. Clones containing the complete genomic sequence for BK virus, JC virus, K virus, polyoma virus, and SV40 are available from American Type Culture Collection, 12301 Parklawn Drive, Rockville, Md. 20852, U.S.A. (ATCC). Clones containing promoters, bacterial origins of replication, and a variety of vectors are also available from the commercial sources listed above or ATCC, as well as other sources well known to those skilled in the art of recombinant DNA manipulation. Specific sequences encoding particular proteins or regulatory sequences may be obtained from these clones using standard recombinant DNA techniques. The particular foreign genes whose expression in mammalian cells is desired, and sources for sequences encoding them, will be readily apparent to those skilled in the art.

### B. Recombinant: Procedures For Vector Construction

Vector construction entails conventional molecular biology, microbiology, and recombinant DNA techniques. Such techniques are well known to one of ordinary skill in the art and are explained fully in the literature. See, e.g., Maniatis, et al., “Molecular Cloning: A Laboratory Manual” (1982); “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames, et al., eds., 1985); “Transcription and Translation” (B. D. Hames, et al., eds., 1984); “Animal Cell Culture” (R. I. Freshney, ed., 1986); “immobilized Cells and Enzymes” (IRL Press, 1986); B. Perbal, “A Practical Guide to Molecular Cloning” (1984), and Sambrook, et al., “Molecular Cloning: a Laboratory Manual” (1989).

DNA segments corresponding to the papovavirus origin of replication, the papovavirus large T antigen coding sequence and the papovavirus early promoter may be obtained from readily available recombinant DNA materials, such as those available from the ATCC, which include BK virus, JC virus, K virus, polyoma virus, and SV40 virus. DNA segments or oligonucleotides having specific sequences can be synthesized chemically or isolated by one of several approaches. The basic strategies for identifying, amplifying and isolating desired DNA sequences as well as assembling them into larger DNA molecules containing the desired sequence domains in the desired order, are well known to those of ordinary skill in the art. See, e.g., Sambrook, et al., (1989); B. Perbal, (1984). Preferably, DNA segments corresponding to the papovavirus origin, large T antigen and early promoter may be isolated individually using the polymerase chain reaction (M. A. Innis, et al., “PCR Protocols: A Guide To Methods and Applications,” Academic Press, 1990). A complete sequence may be assembled from overlapping oligonucleotides prepared by standard methods and assembled into a complete coding sequence. See, e.g., Edge (1981) Nature 292:756; Nambair, et al. (1984) Science 223:1299; Jay, et al. (1984) J. Biol. Chem., 259:6311.

The assembled sequence can be cloned into any suitable vector or replicon and maintained there in a composition that is substantially free of vectors that do not contain the assembled sequence. This provides a reservoir of the assembled sequence, and segments or the entire sequence can be extracted from the reservoir by excising from DNA in the reservoir material with restriction enzymes or by PCR amplification. Numerous cloning vectors are known to those of skill in the art, and the selection of an appropriate cloning vector is a matter of choice (see, e.g., Sambrook, et al., incorporated herein by reference). The construction of vectors containing desired DNA segments linked by appropriate DNA sequences is accomplished by techniques similar to those used to construct the segments. These vectors may be constructed to contain additional DNA segments, such as those encoding foreign genes for gene therapy, bacterial origins of replication to make shuttle vectors (for shuttling between prokaryotic intermediate hosts and mammalian final hosts), etc.

Procedures for construction and expression of mutant proteins of defined sequence are well known in the art. A DNA sequence encoding a known mutant of papovavirus large T antigen can be synthesized chemically or prepared from the wild-type sequence by one of several approaches, including primer extension, linker insertion and PCR (see, e.g., Sambrook, et al.). Alternatively, additional mutants can be prepared by these techniques having additions, deletions and substitutions in the wild-type sequence. In either case, it is preferable to test the mutants to confirm that they are replication-competent and transformation-negative, by the assays described above. Mutant large T antigen protein for testing may be prepared by placing the coding sequence for the polypeptide in a vector under the control of a promoter, so that the DNA sequence is transcribed into RNA and translated into protein in a host cell transformed by this (expression) vector. The mutant large T antigen protein may be produced by growing host cells transfected by an expression vector containing the coding sequence for the mutant T antigen under conditions whereby the polypeptide is expressed. The selection of the appropriate growth conditions is within the skill of the art.

### C. Intermediate Stage Vectors

Preferably the vector containing the replication cassette will also contain a functional bacterial origin of replication and selection markers that function in bacteria (i.e., a shuttle vector). These will allow cloning of the vector in bacteria to provide a stable reservoir of the vector for storage and to facilitate amplification, where large quantities of the vector containing the replication cassette and any associated foreign genes can be recovered from bacterial culture. The procedures, as well as appropriate bacterial origins and selection markers are well known in the art (see, e.g. Sambrook, et al.). Alternatively, mammalian viral vectors may be amplified in mammalian cell culture, using well known techniques. Appropriate procedures for storage and standardization of preparations containing virus vectors or bacterial cells harboring shuttle plasmid vectors will be readily apparent to those skilled in the art.

### D. Functional Tests of the Vector

Vectors containing the replication cassette of this invention will routinely be tested after they have been constructed to confirm that the vector is replication-competent and non-transforming. These tests will assure that sequences included in the vector do not interfere with the functioning of the replication cassette. Replication competence (i.e., that both the mutant large T antigen and the origin of replication are functional) is usually tested by transfecting a population of non-transformed cells of the target cell type with the vector and monitoring episomal DNA production by Southern blot. Stable transfectants from the replication test can be further tested for soft agar cloning activity or tumorigenesis in nude or SCID mice to confirm that the vector has not transformed the cells. Southern blots of DNA from the stable transfectants may be used to indicate whether they have integrated the vector into genomic DNA or if the vector is being carried as a stable episome.

After a host is introduced with a vector comprising a nucleic acid insert derived from a cDNA library or a genomic library, one or more biochemical or phenotypic changes in the host is determined. The biochemical or phenotypic changes in the host is correlated to the biochemistry or phenotype of a host that lacks the vector. Optionally, the biochemical or phenotypic changes in the infected host organism is further correlated to a host organism that is infected with a viral vector that contains a control nucleic acid of a known sequence in positive sense orientation; the control nucleic acid has similar size but is different in sequence from the nucleic acid insert derived from the library. For example, if the nucleic acid insert derived from the library is identified as encoding a GTP binding protein in positive sense orientation, a nucleic acid derived from a gene encoding green fluorescent protein can be used as a control nucleic acid. Green fluorescent protein is known not be have the same effect as the GTP binding protein when expressed in a host organism.

One of ordinary skill in the art will readily understand that there are many methods to determine phenotypic or biochemical change in a host and to determine the function of a nucleic acid, once the nucleic acid is expressed in a host. In a preferred embodiment, the phenotypic or biochemical trait may be determined by observing phenotypic changes in a host by methods including visual, morphological, macroscopic or microscopic analysis. For example, growth change such as stunting or color change is easily visualized. In another embodiment, the phenotypic or biochemical trait may be determined by complementation analysis, that is, by observing the endogenous gene or genes whose function is replaced or augmented by introducing the nucleic acid of interest. In a third embodiment, the phenotypic or biochemical trait may be determined by analyzing the biochemical alterations in the accumulation of substrates or products from enzymatic reactions according to any means known by one of ordinary skill in the art. In a fourth embodiment, the phenotypic or biochemical trait may be determined by observing any changes in biochemical pathways that may be modified in a host as a result of expression of the nucleic acid. In a fifth embodiment, the phenotypic or biochemical trait may be determined utilizing techniques known by those skilled in the art to observe inhibition of endogenous gene expression in the cytoplasm of cells as a result of expression of the nucleic acid. In a sixth embodiment, the phenotypic or biochemical trait may be determined utilizing techniques known by those skilled in the art to observe changes in the RNA or protein profile as a result of expression of the nucleic acid. In a seventh embodiment, the phenotypic or biochemical trait may be determined by selection of hosts capable of growing or maintaining viability in the presence of noxious or toxic substances, such as, for example, pharmaceutical ingredients.

Phenotypic changes in animals such as mice include change of size, structure, growth rate, mobility, ability to reproduce, number of offspring, response to excipients, development, resistance to pathogens, food consumption, behavior such as socialization and life span. Phenotypic or biochemical changes in cell lines include changes of doubling rate, shape, size, kinase activity, cytokine release, response to excipients (e.g. toxic compounds, pathogens, etc.), division of cell culture, serum-free growth, activation of gene (detected by mRNA and protein profile), and expression of receptor (detected by a biochemical method or an immunoassay using antibodies). Other examples include the production of important proteins or other products for commercial use, such as lipase, melanin, pigments, alkaloids, antibodies, hormones, pharmaceuticals, antibiotics and the like.

Biochemical changes can also be determined by analytical methods, for example, in a high-throughput, fully automated fashion using robotics. Suitable biochemical analysis may include matrix-assisted laser desorption time of flight mass spectrometry (MALDI-TOF), LC/MS, GC/MS, two-dimensional IEF/SDS-PAGE, ELISA or other methods of analyses. The clones in the viral vector library may then be functionally classified based on metabolic pathway affected or visual/selectable phenotype produced in the organism. This process enables a rapid determination of gene function for unknown nucleic acid sequences of a donor organism as well as a host organism. Furthermore, this process can be used to rapidly confirm function of full-length DNA's of unknown function. Functional identification of unknown nucleic acid sequences in a library of one organism may then rapidly lead to identification of similar unknown sequences in expression libraries for other organisms based on sequence homology. Such information is useful in many aspects including human medicine.

One useful means to determine the function of nucleic acids transfected into a host is to observe the effects of gene silencing. An EST/DNA library from a donor organism, may be assembled into a vector. The nucleic acid sequences in the vector library may then be introduced into host cells which post-transcriptionally silences the homologous target gene. The EST/DNA sequences may be introduced into a vector in either the plus or minus sense orientation, and the orientation can be either directed or random based on the cloning strategy. A high-throughput, automated cloning scheme based on robotics may be used to assemble and characterize the library. The library of EST clones is then introduced into a host organism. The vectors containing the EST/cDNA sequences contributed from the original library may now be present in a sufficiently high concentration in the cytoplasm of host cells such that they cause post-transcriptional gene silencing of the endogenous gene in the host.

The invention provides a method to identify genes involved in the regulation of growth by inhibiting or enhancing the expression of specific endogenous genes using vectors. This invention provides a method to characterize specific genes and biochemical pathways in donor organisms or in host organisms using a vector.

A detailed discussion of some aspects of the “gene silencing” effect is provided in U.S. Pat. No. 5,922,602, the disclosure of which is incorporated herein by reference. RNA can reduce the expression of a target gene through inhibitory RNA interactions with target mRNA that occur in the cytoplasm and/or the nucleus of a cell.

It is known that silencing of endogenous genes can be achieved with homologous sequences from the same family. For example, Kumagai et al., (Proc. Natl. Acad. Sci. USA 92:1679 (1995)) report that the Nicotiana benthamiana gene for phytoene desaturase (PDS) was silenced by transfection with a viral RNA derived from a clone containing a partial tomato (Lycopersicon esculentum) cDNA encoding PDS being in a positive sense orientation. This paper is incorporated here by reference. Kumagai et al. demonstrate that gene encoding PDS from one plant can be silenced by transfecting a host plant with a nucleic acid of a known sequence, namely, a PDS gene, from a donor plant of the same family. The present invention provides a method of silencing a gene in a host organism by introducing into a host a vector comprising a nucleic acid insert derived from a cDNA library or a genomic DNA or RNA library from a donor organism. Different from Kumagai et al, the sequence of the nucleic acid insert in the present invention does not need to be identified or isolated prior to the transfection. Another feature of the present invention is that it provides a method to change the expression of a gene of a non-plant host; the plus sense transcript of one organism results in enhancing or reducing expression of the endogenous gene or multigene family of a host organism from Monera, Protista, Fungi or Animalia.

The sequence of the nucleic acid insert in the cDNA clone or in the viral vector can be determined by a standard method, for example, by dideoxy termination using double stranded templates (Sanger et al., Proc., Natl. Acad. Sci. USA 74:5463-5467 (1977)). Once the sequence of the nucleic acid insert is obtained, the sequence of an entire open reading frame of a gene can be determined by probing filters containing full-length cDNAs from the cDNA library with the nucleic acid insert labeled with radioactive, fluorescent, or enzyme molecules. The sequence of an entire open reading frame of a gene can also be determined by RT-PCR (Methods Mol. Biol. 89:333-358 (1998)).

The present invention also provides a method of isolating a conserved gene from a donor organism. Libraries containing full-length cDNAs from fungi, and animals can be obtained from public and private sources or can be prepared from mRNAs. The cDNAs are inserted in viral vectors or in small subcloning vectors such as pBluescript (Strategene), pUC18, M13, or pBR322. Transformed bacteria are then plated and individual clones selected by a standard method. The bacteria transformants or DNAs are rearrayed at high density onto membrane filters or glass slides. Full-length cDNAs can be identified by probing filters or slides with labeled nucleic acid inserts which result in changes in a host organism. Useful labels include radioactive, fluorescent, or chemiluminescent molecules, enzymes, etc.

Alternatively, genomic libraries containing sequences from fungi, animals and libraries from retroviruses can be obtained from public and private sources, or be prepared from genomic DNAs. BAC clones containing entire genomes have been constructed and organized in a minimal overlapping order. Individual BACs are sheared to fragments and directly cloned into viral vectors. Clones that completely cover an entire BAC form a BAC viral vector sublibrary. Genomic clones can be identified by probing filters containing BACs with labeled nucleic acid inserts which result in changes in a host organism. Useful labels include radioactive, fluorescent, or chemiluminecent molecules, enzymes, etc. BACs that hybridize to the probe are selected and their corresponding BAC viral vectors are used to produce infectious RNAs. Host organisms that are transfected with the BAC sublibrary are screened for a phenotypic or biochemical. Once a change is observed or detected, the inserts from these clones or their corresponding plasmid DNAs are characterized by dideoxy sequencing. This provides a rapid method to obtain the genomic sequence of a donor organism. Using this method, once the DNA sequence in one organism is identified, it can be used to identify conserved sequences of similar function that exist in other libraries. This method speeds up the rate of discovering new genes.

Nucleic acid sequences that may result in changing a host phenotype include those involved in cell growth, proliferation, differentiation and development; cell communication; and the apoptotic pathway. Genes regulating growth of cells or organisms include, for example, genes encoding a GTP binding protein, a ribosomal protein L19 protein, an S18 ribosomal protein, etc. Henry et al. (Cancer Res., 53:1403-1408 (1993)) report that erb B-2 (or HER-2 or neu) gene was amplified and overexpressed in one-third of cancers of the breast, stomach, and ovary; and the mRNA encoding the ribosomal protein L19 was more abundant in breast cancer samples that express high levels of erbB-2. Lijsebettens et al. (EMBO J, 13:3378-3388 (1994)) report that in Arabidopsis, mutation at PFL caused pointed first leaves, reduced fresh weight and growth retardation. PFL codes for ribosomal protein S18, which has a high homology with the rat S18 protein. Genes involved in development of cells or organisms include, for example, homeobox-containing genes and genes encoding G-protein-coupled receptor proteins such as the rhodopsin family. Homeobox genes are a family of regulatory genes containing a common 183-nucleotide sequence (homeobox) and coding for specific nuclear proteins (homeoproteins) that act as transcription factors. The homeobox sequence itself encodes a 61-amino-acid domain, the homeodomain, responsible for recognition and binding of sequence-specific DNA motifs. The specificity of this binding allows homeoproteins to activate or repress the expression of batteries of down-stream target genes. Initially identified in genes controlling Drosophila development, the homeobox has subsequently been isolated in evolutionarily distant animal species, plants, and fungi. Several indications suggest the involvement of homeobox genes in the control of cell growth and, when dysregulated, in oncogenesis (Cillo et al., Exp. Cell Res., 248:1-9 (1999). Other nucleic acid sequences that may result in changes of an organism include genes encoding receptor proteins such as hormone receptors, cAMP receptors, serotonin receptors, and calcitonin family of receptors; and light-regulated DNA encoding a leucine (Leu) zipper motif (Zheng et al., Plant Physiol., 116:27-35 (1998)). Deregulation or alteration of the process of cell growth, proliferation, differentiation and development; cell communication; and the apoptotic pathways may result in cancer. Therefore, identifying the nucleic acid sequences involved in those processes and determining their functions are beneficial to the human medicine; it also provides a tool for cancer research.

A library of human nucleic acid sequences is cloned into vectors. The vectors are applied to the host to obtain infection. Each infected host is grown with an uninfected host and a host infected with a null vector. A null vector will show no phenotypic or biochemical change other than the effects of the virus itself. Each host is observed daily for visual differences between the infected host and its two controls. In each host displaying an observable phenotypic or biochemical change a trait is identified. The donor nucleic acid sequence is identified, the full length gene sequence is obtained and the full length gene in the host is obtained, if a gene from the host is associated with the trait. Both genes are sequenced and homology is determined. A variety of biochemical tests may also be made on the host or host tissue depending on the information that is desired. A variety of phenotypic changes or traits and biochemical tests are set forth in this document. A functional gene profile can be obtained by repeating the process several times.

Large amounts of DNA sequence information are being generated in the public domain, which may be entered into a relational database. Links may be made between sequences from various species predicted to carry out similar biochemical or regulatory functions. Links may also be generated between predicted enzymatic activities and visually displayed biochemical and regulatory pathways. Likewise, links may be generated between predicted enzymatic or regulatory activity and known small molecule inhibitors, activators, substrates or substrate analogs. Phenotypic data from expression libraries expressed in transfected hosts may be automatically linked within such a relational database. Genes with similar predicted roles of interest in other organisms may be rapidly discovered.

Challenging a host with a pathogen comprises exposing the host to a sufficient number, one or more, of the pathogen that is able to cause a diseased state of a host of the same species that has not been introduced to any antigen of the pathogen. The challenge may comprise of introducing the sufficient number of pathogen to the host by aerosol exposure, feeding, intravenous injection or drip, injection, direct application, or the like.

Challenging a host with a cancer cell comprises exposing the host to a sufficient number, one or more, of the cancer cell that is able to cause a tumor or lymphoma in a host of the same species that has not been introduced to any antigen of the cancer cell. The challenge may comprise of introducing the sufficient number of the cancer cell to the host by intravenous injection or drip, injection, direct application, insertion under the skin, or the like.

The present invention also provides a method whereby the library constructed, either from a pathogen or a cancer cell, may first be pooled into batches, in which each batch contains two or more vectors with different inserts. A batch may contain five, ten, twenty, fifty, one hundred, or a unknown number between the numbers cited, or a unknown number between one hundred and one thousand. Each batch introduced into a host. Instead identifying a single insert initially, a batch containing a desired insert, i.e. an insert that contains the sequence of an antigen to be identified, is identified. A batch that is thus identified is then in turn divided into other batches where each new batch contains fewer number of inserts than the original batch identified. The new batches are then introduced into hosts and challenged again to identify the new batch that contains the desired insert. The identified new batch is then further divided and a new round of introduction into hosts and challenged performed. The process carried out till the individual desired insert is identified. This method speeds up the rate of discovering and/or identifying genes.

Antigens identified from cancer cells can be tumor-specific transplantation antigens (TSTAs) or tumor-associated transplantation antigens (TATAs). The TSTAs are unique to tumor cells and do not occur on normal cells in an animal. TSTAs can be chemically or physically induced tumor antigens or virally induced tumor antigens. An example of a chemical that can generate a tumor cell that expresses chemically induced tumor antigens is methylcholanthrene. An example of a physical phenomenon that can generate a tumor cell that expresses physically induced tumor antigens is ultraviolet light. TATAs, which are not unique to tumor cells, can be proteins that are expressed on normal cells during fetal development when the immune system is immature and unable to respond but that normally are not expressed in the adult. TATAs can also be proteins that are normally expressed at extremely low levels on normal cells but are expressed at much higher levels on tumor cells. TATAs can be oncofetal tumor antigens or oncogene proteins as tumor antigens. Examples of oncofetal tumor antigens are alpha-fetoprotein, associated with human liver cancer, and carcinoembryonic antigen, associated with human colorectal cancer. An example of an oncogene protein as tumor antigen is the Neu protein, a growth factor receptor, associated with human breast cancer. See Kuby, Immunology 3d ed., 1997, W. H. Freeman and Co., New York.

Immuno-protection of a host against a pathogen is the reduced ability or inability of the pathogen to reproduce in the host, grow in the host, replicate in the host, produce pathogen products (such as proteins, RNA, or DNA) in the host, increase in numbers in the host, infect any host cell, enter any host cell, produce and/or release toxins into the host, produce one or more signs symptomatic of infection of the host by the pathogen, or kill the host. One of ordinary skill in the art understands that “in the host” may mean “in a host cell”, “in a cell of the host”, or “in a host organism but not necessarily in a cell”. Such reduced abilities or inabilities can be measured, detected, or observed by visual means, by instrument, by biochemical assay, by autopsy, by plate count, by plaque assay, or the like.

Immuno-protection of a host against a cancer cell is the reduced ability or inability of the cancer cell to proliferate in the host, grow in the host, metastasize in the host, cell divide in the host, attach to the host, produce one or more signs symptomatic of the cancer in the host, or kill the host. One of ordinary skill in the art understands that “in the host” may mean “in a host organism but not in a cell” or “on the surface of a host organism”. Such reduced abilities or inabilities can be measured, detected, or observed by visual means, by instrument, by biochemical assay, by autopsy, by plate count, or the like.

In one embodiment of the present invention, the replicon comprises an episomal plasmid containing a papovavirus origin of replication and a papovavirus large T antigen mutant form. Such a vector can be constructed by common molecular biology techniques that are familiar to those skilled in the art. Such vectors can replicate episomally in animal, including human, cells and yield levels of gene expression proportional to their episomal copy number. One object of the invention is to provide a vector which will reproduce episomally in high copy number in a mammalian cell without transforming the cell. Another object of the invention is to provide a method for functional genomic analysis, whereby a library of foreign genes can be imported into said vector, in a plus or minus sense orientation, to affect the proteome or metabolism of the host cell in such a way that a quantitative relationship can be derived linking gene sequence to phenotype and function. Function can be determined by phenotypic observation, through metabolic assays, growth, growth retardation and apoptosis assays, quantitative protein profiling (proteomics) and other assays for phenotypic or metabolic changes familiar to those skilled in the art.

In a preferred embodiment the vector consists of the SV40 or BK virus origin of replication, and a DNA sequence encoding a mutant form of the papovavirus large T antigen which contains a replication-competent binding site for the origin of replication but which is negative for binding to at least one of wild-type p53 or retinoblastoma tumor suppressor (RE) gene products, preferably both, the DNA sequence being operatively linked to a homologous or heterologous promoter. In an alternative embodiment of the vector, the DNA sequence encoding a mutant form of papovavirus large T antigen is operationally linked either to a papovavirus early promoter, to a promoter which is inducible, or to a promoter which is under hormonal control. Such vectors, when introduced into competent host cells, can be shown to replicate for at least 5 months, achieve a high stable copy number (150) without inducing episome-mediated cell death, have a low rate of integration, transcribe genes in proportion to their copy number, can be efficiently transferred to daughter cells during cell division, can be shuttled to bacteria, and can persist in treated host cells for several months without selection pressure. When libraries of genes from same or different organiams are imported into said vectors by a variety of techniques known to those skilled in the art and competent host cells are transfected with said vectors, phenotypic and functional screens can be used to determine the function of various genes in the library by exploiting gene overexpression as well as gene silencing effects.

It should be understood that the promoters and other controlling elements in the replicons described are specified for the purpose of illustrating enabling technology. Other promoters and controlling elements could be used to provide the vector with a wider cell or tissue host range (eg., normal host cells or tissues vs. cancerous or diseased host cells or tissues; non-growing host cells or tissues vs. growing host cells or tissues; non-mammalian host cells, tissues or organisms vs. mammalian host cells, tissues or organisms, etc.).

An embodiment of this invention involves the treatment of the previously described compositions (see U.S. Pat. Nos. 5,844,107 and 6,008,336) with reagents that condense the DNA to render the vector more efficiently transportable into the host cells and specifically into the nucleus of the host cells. The condensation of nucleic acids to import them efficiently into cells is a method that is inherently different than traditional methods that generally rely on physical transport of exogenous genes (including naked normal or relaxed DNA) into mammalian cells, which include transfection, direct microinjection, electroporation, ballistic injection, and coprecipitation with calcium phosphate. The novel nucleic acid delivery method involves compaction of nucleic acid without aggregation. The compacted nucleic acid can be shown to efficiently cross the membranes of living cells, including a cell in a multicellular organism. The DNA condensed into small particles may be more suitable for nuclear translocation through the nuclear pores and my be protected against nucleases. When the nucleic acid so treated includes an expressible gene, or a library of plus or minus sense oriented genes, the gene(s) can be expressed in the cell.

The method to condense nucleic acid consists of exposing the nucleic acid to a polycationic carrier, such as poly-lysine, with or without a chaotropic salt, preferably NaCl, which may be added to prevent nucleic acid aggregation. The condensed nucleic acid will preferably consist of monomolecular toroids, with diameters ranging from 12 to 100 nm. Compacted nucleic acid replicons are less susceptible to degradation once inside the host cell than normal, relaxed or aggregated nucleic acids. Such condensates can enter host cells efficiently via pinocytocis, and pass through the nuclear membrane through similar mechanisms or through pores in the nuclear matrix. The carrier/nucleic acid complexes dissociate to allow replication of the vector to high copy number and subsequent gene transcription. In some embodiments of the invention, a tissue-specific carrier molecule can be prepared, consisting of a bifunctional molecule having a nucleic acid-binding moiety and a target tissue-binding noiety. The nucleic acid is then compacted at high concentrations with the carrier molecule at a critical salt concentration. The nucleic acid-loaded carrier molecule bears a single nucleic acid molecule, which can target the nucleic acid specifically to desired cells or tissues.

Thus, such compacted nucleic acids, with or without cell- or tissue-targeting carriers, can be used to express libraries of genes each individually imported by a single carrier molecule. By relating expression of the genes in a library with functional phenotypic, proteomic or metabolic screens, gene function can be determined. Such a method of use of the invention enables functional genomic applications.

In another embodiment, the genes of foreign organisms (such as microbial or viral pathogens) can be safely expressed in a library (expression library) in such compacted nucleic acid replicons and which, when introduced into an animal, with or without cell- or tissue-specific targeting, will produce a library of antigens in the animal. When challenged with the foreign organism, protected animals can be identiied and the antigen(s) responsible for protection correlated to the specific genes in the library (antigen discovery). When this approach is used interactively with several candidate protective antigens, the best antigen for vaccine development may be identified (antigen prototyping).

Another embodiment of the invention is to use the previously described nucleic acid compositions to express libraries of genes from diseased cells or tissue. For example, the expression immunization approach described in the previous paragraph can be adapted to immunize animals with cancer cell-derived libraries, or with preselected genes therefrom, to identify and prototype cancer antigens. This approach could inherently be used to identify unique antigens associated with any diseased cell or tissue and to differentiate those novel antigens from normal cell or tissue phenotypes.

These new applications to functional genomics and antigenics of compacted non-viral nucleic acid replicon technology are described generally to illustrate such new uses for previously described compositions. The above mentioned descriptions are not meant to limit the present invention by suggesting that additional new compositions or improvements could not be developed, nor that new uses for this technology, or collections of technologies, could not be found. Specific examples for vector construction and use have been cited and it is generally understood that those skilled in the art will find such references adequate to carry out the present invention.

### EXAMPLES

### Example 1

The goal of this example is to identify a gene encoding a negative growth regulator that when expressed would inhibit growth of a cell line such as HT-1376.

A human genomic library is constructed by taking purified human genomic DNA and performing a partial BamHI (or any other restriction enzyme that has a unique site in pRB-cneoX) digest. The DNA products of the partial digest are size separated using agarose gel electrophoresis and DNA fragments ranging from 4 kb to 16 kb (or any other appropriate range of sizes) are isolated. These isolated fragments are then cloned into the BamHI unique restriction site (or any other restriction site depending on which restriction enzyme was used to construct the library) of the pRB-cneoX episome. Alternatively, pRB-cneoX can be modified by linking a strong constitutive promoter near the unique restriction site.

Individual members of the library are then introduced into a HT-1376 cell line using the method taught in U.S. Pat. No. 5,624,820 such that no more than one member of the library is introduced into each HT-1376 cell. For each member of the library, 0.1 μg of plasmid DNA and 0.4 μg of lipofectin in 30 μl of Optimem (Gibco-Bethesda Research Labs, Gaithersburg, Md.) can be used to transfect 1.5×10⁴ cells. After 6 hours of incubation, DMEM is added with supplemental fetal calf serum to obtain a concentration of 10%. Three days after transfection, 200 μg/ml G418 is added to the media to initiate selection. The amount of DNA, lipofectin, and number of cells can be proportionately increased to ensure that at least one episome enters one cell per transfection. Individual members are then screened for a phenotypic change or trait characterized by non-growth of the cell in terms of few or no cell divisions over a specific period of time. The desired phenotypic change is compared to a HT-1376 cell line that contains the pRB-cneoX episome without any insert.

The cell lines identified with the desired phenotypic change are selected and the insert is amplified through polymerase chain reaction (PCR) using a pair of oligonucleotide primers wherein one primer is complementary to the sequence of pRB-cneoX on side of the unique restriction site and the other primer is complementary to the sequence of pRB-cneoX on the opposite strand on the opposite side of the unique restriction site. One of ordinary skill in the art can design such primers and can further incorporate further features such as other unique restriction sites at the ends of the primer to facilitate cloning of the PCR product into any suitable eukaryotic or prokaryotic vector.

The insert can be cloned into a suitable vector, such as pBR322, in order to be amplified in a suitable organism, such as E. coli. With sufficient quantities of the insert, it can be sequenced using the dideoxy chain termination method. With its complete nucleotide sequence determined, the structure of the gene is determined and the predicted amino acid sequence can also be determined.

The method can be automated into a high-throughput format whereby the cells are grown in prearranged wells on a plate.

### Example 2

The goal of this example is to identify a nucleic acid sequence encoding an antigen, of a pathogen, that when expressed by a host organism and presented to the immune system of the host organism would confer on the host organism immunity to the pathogen.

A genomic library of Yersinia pestis is constructed by taking purified Yersinia pestis genomic DNA and performing a partial BamHI (or any other restriction enzyme that has a unique site in pRB-cneoX) digest. The DNA products of the partial digest are size separated using agarose gel electrophoresis and DNA fragments ranging from 0.1 kb to 4 kb (or any other appropriate range of sizes) are isolated. These isolated fragments are then cloned into the BamHI unique restriction site (or any other restriction site depending on which restriction enzyme was used to construct the library) of a pRB-cneoX episome that is modified by linking a strong constitutive mammalian promoter near the unique restriction site.

Individual members of the library are then introduced into a rat using the method taught in U.S. Pat. No. 6,008,336 such that no more than one member of the library is introduced into each rat. Each rat, such as an adult male Sprague-dawley rat, or any rat susceptible to infection by Yersinia pestis, approximately 250 g in weight, is anesthetized with ether. 300-400 μl of a solution containing 300 μg of a member of the pRB-cneoX-based library complexed with galactose-poly-L-lysine, is infused into the caudal cava vein. The complex is prepared by: vortexing a solution of 300 μg of DNA in 200 μl of 0.75 M NaCl (added from a 5 M NaCl solution) at medium speed (using a VIBRAX machine, IKA-VIBRAX-VXR), adding to the solution 84 μg of poly-L-lysine-galactose in 200 μl of 0.75 M NaCl (added from a 5 M NaCl solution) dropwise over a period of 30 minutes to 1 hour in 20 μl aliquots, to the turbid solution is added 3 μl of 5 M NaCl into the vortexing solution until the turbidity disappears as monitored by eye, and then the solution is subjected to CD spectroscopic monitoring while 2 μl aliquots of 5 M NaCl are gradually added until the diagnostic spectrum of the DNA complex is observed. (See U.S. Pat. No. 6,008,336 for a detailed iteration of the method.) Rats are then allowed express any peptide encoded in the insert. Rats are then exposed to Yersinia pestis to allow infection to take place. Rats that are immune to Yersinia pestis infection are identified.

The rats identified with immunity to Yersinia pestis are selected and the insert is amplified through PCR using a pair of oligonucleotide primers wherein one primer is complementary to the sequence of pRB-cneoX on side of the unique restriction site and the other primer is complementary to the sequence of pRB-cneoX on the opposite strand on the opposite side of the unique restriction site. One of ordinary skill in the art can design such primers and can further incorporate further features such as other unique restriction sites at the ends of the primer to facilitate cloning of the PCR product into any suitable eukaryotic or prokaryotic vector.

The insert can be cloned into a suitable vector, such as pBR322, in order to be amplified in a suitable organism, such as E. coli. With sufficient quantities of the insert, it can be sequenced using the dideoxy chain termination method. With its complete nucleotide sequence determined, the predicted amino acid sequence of the peptide/antigen can also be determined.

Such a peptide is an antigen of Yersinia pestis that when presented to the immune system of a rat is able to confer immunity to the rat against Yersinia pestis. Such antigens can be further investigated in order that an antigen suitable for conferring immunity on humans is developed.

### Example 3

The goal of this example is to identify a nucleic acid sequence encoding a cancer antigen, of a cancer cell of a first tumor, that when expressed by a host organism and presented to the immune system of the host organism would confer on the host organism immunity to decreased growth or transplantation rejection of a second tumor that is introduced to the host organism.

A cDNA library of a murine fibrosarcoma cell is constructed by taking purified mRNA preparation of a murine fibrosarcoma cell and constructing a cDNA using reverse transcriptase. A subtractive hybridization step may be performed to using cDNA constructed from a normal murine xxx cell in order to enrich the cDNA library of the murine fibrosarcoma cell to cDNA generated from the murine fibrosarcoma cell but not generated from the normal murine xxx cell. The subtracted cDNA of the murine fibrosarcoma cell is then cloned into a unique restriction site in a modified pRB-cneoX so that each vector only has one cDNA insert. The pRB-cneoX episome has been modified by linking a strong constitutive mammalian promoter near the unique restriction site.

Individual members of the library are then introduced into BALB/c mice, using the method recited in Example 2 and taught in U.S. Pat. No. 6,008,336 such that no more than one member of the library is introduced into each animal. Animals are then set aside for four weeks to allow expression of any peptide encoded in the insert. Murine fibrosarcoma cells are introduced into the animals insertion under the skin in the mid-dorsum (see Carbone et al., 1991, Int. J. Cancer 47:619-25). Mice that do not support tumor growth are identified.

Mice identified as not supporting tumor growth are selected and the insert is amplified through PCR using a pair of oligonucleotide primers wherein one primer is complementary to the sequence of pRB-cneoX on side of the unique restriction site and the other primer is complementary to the sequence of pRB-cneoX on the opposite strand on the opposite side of the unique restriction site. One of ordinary skill in the art can design such primers and can further incorporate further features such as other unique restriction sites at the ends of the primer to facilitate cloning of the PCR product into any suitable eukaryotic or prokaryotic vector.

The insert can be cloned into a suitable vector, such as pBR322, in order to be amplified in a suitable organism, such as E. coli. With sufficient quantities of the insert, it can be sequenced using the dideoxy chain termination method. With its complete nucleotide sequence determined, the predicted amino acid sequence of the peptide/antigen can also be determined.

Such a peptide is a tumor-specific transplantation antigen of the murine fibrosarcoma that when presented to the immune system of a mouse is able to mount a cell-mediated response to the cancer cell(s). Such tumor-specific transplantation antigens can be further investigated in order that similar tumor-specific transplantation antigens suitable for humans to be developed.

Although the invention has been described with reference to the presently preferred embodiments, it should be understood that various modifications can be made without departing from the spirit of the invention.

All publications, patents, patent applications, and web sites are herein incorporated by reference in their entirety to the same extent as if each individual patent, patent application, or web site was specifically and individually indicated to be incorporated by reference in its entirety.

What is claimed is:

1. A method of compiling a functional gene profile of a donor organism, comprising:
(a) introducing into an episomal non-transforming non-viral vector a mixture of a donor organism derived DNA or RNA sequences to construct an episomal non-transforming non-viral vector-based library, wherein the sequences are unidentified, wherein each member of the library comprises an insert from the mixture;
(b) introducing into a host said one or more members of the library;
(c) transiently expressing said unidentified nucleic acid in the host;
(d) determining one or more phenotypic or biochemical changes in the host;
(e) identifying an associated trait relating to said one or more phenotypic or biochemical changes;
(f) identifying the member that results in said one or more changes in the host;
(g) repeating steps (b)-(f) until at least one nucleic acid sequence associated with said trait is identified, whereby a functional gene profile of the host or of the donor organism is compiled.

2. A method of identifying the sequence of an antigen of a pathogen, which when expressed in a host confers immuno-protection on the host against the pathogen, comprising:
(a) introducing into an episomal non-transforming non-viral vector a mixture of the pathogen derived DNA or RNA sequences to construct an episomal non-transforming non-viral vector-based library, wherein each member of the library comprises an insert from the mixture;
(b) introducing the library into a group of hosts wherein each host contains one member;
(c) expressing each insert, capable of expression in the host, in the host in which the member resides;
(d) challenging each of the host with the pathogen;
(e) determining which host has immuno-protection against the pathogen; and
(f) determining the sequence of the insert in the host determined in step (e);
whereby the sequence of the antigen of the pathogen is identified.

3. A method of identifying the sequence of an antigen of a cancer cell, which when expressed in a host confers immuno-protection on the host against the cancer cell, comprising:
(a) introducing into an episomal non-transforming non-viral vector a mixture of the cancer cell derived DNA sequences to construct an episomal non-transforming non-viral vector-based library, wherein each member of the library comprises an insert from the mixture;
(b) introducing the library into a group of hosts wherein each host contains one member;
(c) expressing each insert, capable of expression in the host, in the host in which the member resides;
(d) challenging each of the host with the cancer cell;
(e) determining which host has immuno-protection against the cancer cell; and
(f) determining the sequence of the insert in the host determined in step (e);
whereby the sequence of the antigen of the cancer cell is identified.

4. The method according to claim 1, wherein the episomal non-transforming non-viral vector comprises a replication-competent, transformation-negative vector comprising at least one papovavirus origin of replication, a first DNA sequence encoding a mutant form of papovavirus large antigen which contains a replication-competent binding site for the origin of replication and which is negative for binding to and to retinoblastoma tumor suppressor gene product due to a mutation in a codon in the p53 binding domain of the large T antigen and a mutation in a codon in the RB binding domain of the large T antigen, the DNA sequence being operatively linked to a first promoter which is functional in the host cell, and a second DNA sequence encoding the foreign gene operatively linked to a second promoter which is function in the host.

5. The method according to claim 4, wherein the papovavirus origin of replication is a BK virus origin of replication or a SV40 origin of replication.

6. The method according to claim 5, wherein the mutant form of the large T antigen contains a replication competent binding site for both the BK virus origin of replication and the SV40 origin of replication.

7. The method according to claim 4, wherein the replication-competent, transformation-negative vector further comprises a bacterial origin of replication.

8. The method according to claim 4, wherein the first promoter is inducible.

9. The method according to claim 4, wherein the first promoter is constitutive.

10. The method according to claim 4, wherein the first promoter is under hormonal control.

11. The method according to claim 1, wherein the episomal non-transforming non-viral vector comprises a DNA sequence encoding a mutant form of SV40 large T antigen which (a) contains a replication-competent binding site for SV40 origin of replication and (b) is negative for binding to wild-type p53 and to retinoblastoma tumor suppressor gene product due to a mutation in a codon in the p53 binding domain of the large T antigen and a mutation in a codon in the RB binding domain of the large T antigen.

12. The method according to claim 11, wherein residue 107 of the mutant SV40 large T antigen is lysine and residue 402 is glutamic acid.

13. The method according to claim 11, wherein the mutant form of SV40 large T-antigen also contains a replication-competent binding site for a BK virus origin of replication.

14. The method according to claim 1, wherein prior to step (b) is the step: compacting each member of the library with a carrier in the presence of a chaotropic salt to a diameter of less than 30 nm, wherein the carrier comprises a target binding moiety conjugated to a nucleic acid binding moiety, wherein the target binding moiety comprises an antibody or a specific binding fragment thereof which binds to a secretary component of a mammalian polymeric immunoglobulin receptor, where the nucleic acid binding moiety comprises of a polycationic polymer comprising positively charged amino acids.

15. The method according to claim 14, wherein each member of the library comprises a promoter operably linked to an oligonucleotide encoding one or more gene product encoded in the insert.

16. The method according to claim 15, wherein the promoter is a viral promoter.

17. The method according to claim 16, wherein the viral promoter is selected from the group consisting of the SV40 promoter, the MMTV promoter, and the CMV promoter.

18. The method according to claim 14, wherein the target binding moiety is an antibody.

19. The method according to claim 18, wherein the antibody is a monoclonal antibody.

20. The method according to claim 14, wherein the polycationic polymer comprising positively charged amino acids is poly-L-lysine.

21. The method according to claim 1, wherein prior to step (b) is the step: mixing one or more members of the library with a carrier molecule at a chaotropic salt concentration sufficient for compaction of a complex consisting essentially of a single molecule of one member and a sufficient number of carrier molecules to provide a charge ratio of 1:1, in the form of a condensed sphere, whereby unaggregated complexes are formed, wherein each complex consists essentially of a single molecule of a member and one or more carrier molecules.

22. The method of claim 21, wherein the chaotropic salt is NaCl.

23. The method of claim 22, wherein the member and the carrier molecule are each, at the time of mixing, in a solution having a salt concentration of 0.05 to 1.5 M.

24. The method of claim 21, wherein the carrier molecule is a polycation and the molar ratio of the phosphate groups of the member to the positively charged groups of the polycation is in the range of 4:1 to 1:4.

25. The method of claim 24, wherein the polycation is added slowly to the members, while vortexing at high speed.

26. The method of claim 21, in which formation of the complexes is monitored to detect, prevent or correct, the formation of aggregated or relaxed complexes.

27. The method of claim 26, wherein formation of the complexes is monitored by a method selected from the group consisting of electron microscopy, circular dichroism, and absorbance measurement.

28. The method of claim 21, further comprising the step of: complexing the unaggregated complexes with lipids.

29. The method of claim 21, wherein the theoretical minimum diameter is calculated using partial specific volume.

30. The method of claim 21, wherein the theoretical minimum diameter is calculated using X-ray diffraction density.

31. The method of claim 21, wherein diameter of the complex is measured using uranyl acetate staining and electron microscopy.

32. The method of claim 21, wherein the carrier molecule comprises a target cell binding moiety.

33. The method of claim 21, wherein the carrier molecule comprises a target cell binding moiety covalently linked to a nucleic acid binding moiety.
